SEQUENCE LISTING (32 sequences)

SEQ ID NO: 1 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 2 - DNA, 2261 bp, Homo sapiens
gaaatcaggctccgggccggccgaagggcgcaactttcccccctcggcgccccaccggctcccgcgcgcctcccctcgcgcccgagcttcgagccaagcagcgtcctggggagcgcgtcatggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaaccctgtgcaacagccactacattacttcaaactgagatccttccttttgagggagcaagtccttccctttcattttttccagtcttcctccctgtgtattcattctcatgattattattttagtgggggcggggtgggaaagattactttttctttatgtgtttgacgggaaacaaaactaggtaaaatctacagtacaccacaagggtcacaatactgttgtgcgcacatcgcggtagggcgtggaaaggggcaggccagagctacccgcagagttctcagaatcatgctgagagagctggaggcacccatgccatctcaacctcttccccgcccgttttacaaagggggaggctaaagcccagagacagcttgatcaaaggcacacagcaagtcagggttggagcagtagctggagggaccttgtctcccagctcagggctctttcctccacaccattcaggtctttctttccgaggcccctgtctcagggtgaggtgcttgagtctccaacggcaagggaacaagtacttcttgatacctgggatactgtgcccagagcctcgaggaggtaatgaattaaagaagagaactgcctttggcagagttctataatgtaaacaatatcagactttttttttttataatcaagcctaaaattgtatagacctaaaataaaatgaagtggtgagcttaaccctggaaaatgaatccctctatctctaaagaaaatctctgtgaaacccctatgtggaggcggaattgctctcccagcccttgcattgcagaggggcccatgaaagaggacaggctacccctttacaaatagaatttgagcatcagtgaggttaaactaaggccctcttgaatctctgaatttgagatacaaacatgttcctgggatcactgatgactttttatactttgtaaagacaattgttggagagcccctcacacagccctggcctctgctcaactagcagatacagggatgaggcagacctgactctcttaaggaggctgagagcccaaactgctgtcccaaacatgcacttccttgcttaaggtatggtacaagcaatgcctgcccattggagagaaaaaacttaagtagataaggaaataagaaccactcataattcttcaccttaggaataatctcctgttaatatggtgtacattcttcctgattattttctacacatacatgtaaaatatgtctttcttttttaaatagggttgtactatgctgttatgagtggctttaatgaataaacatttgtagcatcctctttaatgggtaaacagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - protein, 198 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAGNRRRVCKCPRPVVKSGDKPSLSARYV

SEQ ID NO: 4 - DNA, 2150 bp, Homo sapiens
gaaatcaggctccgggccggccgaagggcgcaactttcccccctcggcgccccaccggctcccgcgcgcctcccctcgcgcccgagcttcgagccaagcagcgtcctggggagcgcgtcatggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagggaaccgaagacgtgtttgcaaatgtccccggcctgtggtcaaatcgggagacaagcccagcctttcggcgagatacgtctaaccctgtgcaacagccactacattacttcaaactgagatccttccttttgagggagcaagtccttccctttcattttttccagtcttcctccctgtgtattcattctcatgattattattttagtgggggcggggtgggaaagattactttttctttatgtgtttgacgggaaacaaaactaggtaaaatctacagtacaccacaagggtcacaatactgttgtgcgcacatcgcggtagggcgtggaaaggggcaggccagagctacccgcagagttctcagaatcatgctgagagagctggaggcacccatgccatctcaacctcttccccgcccgttttacaaagggggaggctaaagcccagagacagcttgatcaaaggcacacagcaagtcagggttggagcagtagctggagggaccttgtctcccagctcagggctctttcctccacaccattcaggtctttctttccgaggcccctgtctcagggtgaggtgcttgagtctccaacggcaagggaacaagtacttcttgatacctgggatactgtgcccagagcctcgaggaggtaatgaattaaagaagagaactgcctttggcagagttctataatgtaaacaatatcagactttttttttttataatcaagcctaaaattgtatagacctaaaataaaatgaagtggtgagcttaaccctggaaaatgaatccctctatctctaaagaaaatctctgtgaaacccctatgtggaggcggaattgctctcccagcccttgcattgcagaggggcccatgaaagaggacaggctacccctttacaaatagaatttgagcatcagtgaggttaaactaaggccctcttgaatctctgaatttgagatacaaacatgttcctgggatcactgatgactttttatactttgtaaagacaattgttggagagcccctcacacagccctggcctctgctcaactagcagatacagggatgaggcagacctgactctcttaaggaggctgagagcccaaactgctgtcccaaacatgcacttccttgcttaaggtatggtacaagcaatgcctgcccattggagagaaaaaacttaagtagataaggaaataagaaccactcataattcttcaccttaggaataatctcctgttaatatggtgtacattcttcctgattattttctacacatacatgtaaaatatgtctttcttttttaaatagggttgtactatgctgttatgagtggctttaatgaataaacatttgtagcatcctctttaatgggtaaacagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - protein, 198 aa, Pongo pygmaeus
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPVHTRGLDFACDIYIWAPLAGTCGVLLLSLVITLYCNHRNRRRVCKCPRPVVKSGGKPSLSERYV

SEQ ID NO: 6 - DNA, 597 bp, Pongo pygmaeus
atggccttacccgtgaccgccttgctcctgccgctggccttgctgctccacgccgccaggccgagccagttccgggtgtcgccgctggatcggacctggaacctgggcgagacggtggagctgaagtgccaggtgctgctgtccaacccgacgtctggctgctcctggctcttccagccgcgtggcgccgccgccagtcccaccttcctcctatacctctcccaaaacaagcccaaggcggccgaggggctggacacccagcggttctcgggcaagaggttgggggacaccttcgtcctcaccctgagcgacttccgccgggagaacgaaggctactatttctgctcggccctgagcaactccatcatgtacttcagccacttcgtgccggtcttcctgccagtgcacacgagggggctggacttcgcctgtgatatctacatctgggcgcccttggccgggacctgtggggtccttctcctgtcactggttatcaccctttactgcaaccacaggaaccgaagacgtgtttgcaaatgtccccggcctgtggtcaaatctggaggcaagcccagcctttcggagagatatgtctaa

SEQ ID NO: 7 - protein, 310 aa, Mus musculus
MASPLTRFLSLNLLLLGESIILGSGEAKPQAPELRIFPKKMDAELGQKVDLVCEVLGSVSQGCSWLFQNSSSKLPQPTFVVYMASSHNKITWDEKLNSSKLFSAMRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVALLLSLIITLICYHRSRKRVCKCPSIACLCLKLQGSKWYESVICSALAVSIRCNKSKSGELPLAVHLDIRAPCKNWEIAGSLVERYGKSGKHSPLSLKAVVESN

SEQ ID NO: 8 - DNA, 933 bp, Mus musculus
atggcctcaccgttgacccgctttctgtcgctgaacctgctgctgctgggtgagtcgattatcctggggagtggagaagctaagccacaggcacccgaactccgaatctttccaaagaaaatggacgccgaacttggtcagaaggtggacctggtatgtgaagtgttggggtccgtttcgcaaggatgctcttggctcttccagaactccagctccaaactcccccagcccaccttcgttgtctatatggcttcatcccacaacaagataacgtgggacgagaagctgaattcgtcgaaactgttttctgccatgagggacacgaataataagtacgttctcaccctgaacaagttcagcaaggaaaacgaaggctactatttctgctcagtcatcagcaactcggtgatgtacttcagttctgtcgtgccagtccttcagaaagtgaactctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaagcgtgtttgcaaatgtcccagtatagcatgcttgtgcctcaaactgcaaggaagcaagtggtatgaatctgtgatctgctcagctctggctgtgagcatcagatgtaacaaatcaaagtcaggagaactgcctttagcggtgcacctggacatcagagccccttgtaagaactgggaaattgctggcagtctagtggagcggtacggtaaatctggaaaacactcccctctgtcactgaaggctgtagtagaatccaattaa

SEQ ID NO: 9 - protein, 207 aa, Mus musculus
MDAELGQKVDLVCEVLGSVSQGCSWLFQNSSSKLPQPTFVVYMASSHNKITWDEKLNSSKLFSAMRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVALLLSLIITLICYHRSRKRVCKCPRPLVRQEGKPRPSEKIV

SEQ ID NO: 10 - DNA, 1452 bp, Mus musculus
cgttgacccgctttctgtcgctgaacctgctgctgctgggtgagtcgattatcctggggagtggagaagctaagccacaggcacccgaactccgaatctttccaaagaaaatggacgccgaacttggtcagaaggtggacctggtatgtgaagtgttggggtccgtttcgcaaggatgctcttggctcttccagaactccagctccaaactcccccagcccaccttcgttgtctatatggcttcatcccacaacaagataacgtgggacgagaagctgaattcgtcgaaactgttttctgccatgagggacacgaataataagtacgttctcaccctgaacaagttcagcaaggaaaacgaaggctactatttctgctcagtcatcagcaactcggtgatgtacttcagttctgtcgtgccagtccttcagaaagtgaactctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaagcgtgtttgcaaatgtcccaggccgctagtcagacaggaaggcaagcccagaccttcagagaaaattgtgtaaaatggcaccgccaggaagctacaactactacatgacttcagatctcttcttgcaagaggccaggccctcctttttcaagtttcctgctgtcttatgtattgccctctgtattgttttagtaggggtgtgatggggacagttcctttttctttatgaattctctttgacacaaagcatacttgtatgcatacaatgggagtaatgagcagactgtaacaccagagctagttccagtttcggggtccatgtcgctggtggcctcagcacccacttgatataaatctcctgtctgcccatcatatagaagaagctgaagatcagaggtggaaacagcaggatctgtagacccggagagaacccaagctagaggaaccctcactgactggtgcagggatctcacccccatcccctgagctctctgtttaggtatgtgtctttagtatagcatgcttgtgcctcaaactgcaaggaagcaagtggtatgaatctgtgatctgctcagctctggctgtgagcatcagatgtaacaaatcaaagtcaggagaactgcctttagcggtgcacctggacatcagagccccttgtaagaactgggaaattgctggcagtctagtggagcggtacggtaaatctggaaaacactcccctctgtcactgaaggctgtagtagaatccaattaaagctattcaaaccacaaaaaaaaaaaaaaaaaaaaaa

SEQ ID NO: 11 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 12 - DNA, 744 bp, Mus musculus
atggcctcaccgttgacccgctttctgtcgctgaacctgctgctgatgggtgagtcgattatcctggggagtggagaagctaagccacaggcacccgaactccgaatctttccaaagaaaatggacgccgaacttggccagaaggtggacctggtatgtgaagtgttggggtccgtttcgcaaggatgctcttggctcttccagaactccagctccaaactcccccagcccaccttcgttgtctatatggcttcatcccacaacaagataacgtgggacgagaagctgaattcgtcgaaactgttttctgccgtgagggacacgaataataagtacgttctcaccctgaacaagttcagcaaggaaaacgaaggctactatttctgctcagtcatcagcaactcggtgatgtacttcagttctgtcgtgccagtccttcagaaagtgaactctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccctctgctgtccttgatcatcactctcatctgctaccacaggagccgaaagcgtgtttgcaaatgtcccaggccgctagtcagacaggaaggcaagcccagaccttcagagaaaattgtgtaa

SEQ ID NO: 13 - protein, 236 aa, Rattus norvegicus
MASRVICFLSLNLLLLDVITRLQVSGQLQLSPKKVDAEIGQEVKLTCEVLRDTSQGCSWLFRNSSSELLQPTFIIYVSSSRSKLNDILDPNLFSARKENNKYILTLSKFSTKNQGYYFCSITSNSVMYFSPLVPVFQKVNSIITKPVTRAPTPVPPPTGTPRPLRPEACRPGASGSVEGMGLGFACDIYIWAPLAGICAVLLLSLVITLICCHRNRRRVCKCPRPLVKPRPSEKFV

SEQ ID NO: 14 - DNA, 1010 bp, Rattus norvegicus
ccctagagccctagcttgacctaaggtgctggtgggacgcacaccatggcctcacgggtgatctgctttctgtcgctgaacctgctactgctggatgttatcactaggctccaggtttccggacagttacagttgtcaccaaagaaagtggacgctgaaattggccaggaggtgaagctaacatgcgaagtgctgcgggacacttcgcaaggatgctcttggctcttccggaactccagctccgaactcctccagcccaccttcatcatctatgtatcttcatcccggagcaagctgaacgatatactggatccgaatctgttctctgcccggaaggaaaacaacaaatacatcctcaccctgagcaagttcagcactaaaaaccaaggctactatttctgctcaatcaccagcaactcggtgatgtacttcagtcctctggtgccggtgtttcagaaagtgaactctattatcaccaagccggtgacgcgagctcccacaccagtgcctcctcctacagggacaccccggcccctacgaccagaagcttgccgacccggggcgagtggctcagtggagggaatgggattgggcttcgcctgcgatatttacatctgggcacccttggccggaatctgcgcggttcttctgctgtccctggtcatcactctcatctgctgccacaggaaccgaaggcgtgtttgcaaatgtcccaggccccttgtcaagcccagaccttcagagaaattcgtgtaaaatggcgccactaggaagccacaactactacatgacttcagagatttctcacaagagaccgggccctcctttttcagagtttcctgctggcttatatattgtcctctgtattgttttaggggtaggatggggacagttcctttttctttatgaattctctttgatacaaaacatacttgtatgcacacaatggggtaaagatcagactgtaacaccagagatagtcccagtttcagggtcagcgtagctggtgg

SEQ ID NO: 15 - protein, 237 aa, Cavia porcellus
MAPRGSAWLLLLPVALLLDAATAQGASQFRMSPRELVAQVGTKVTLRCEVLVPNAPAGCSWLFQPRHDAKGPTFLLYHSASGTKLAPGLEQKRFSPSKSSNTYTLTVNSFQKRDEGYYFCSVSGNMMLYFSPFVPVFLPAPRTTTPPPPPTTPTPSVQPTSVRPETCVVSKGAAGARWLDLSCDVYIWAPLASTCAALLLALVITIICHRRNRQRVCKCPRPQARSGGKPSPSGKLV

SEQ ID NO: 16 - DNA, 1330 bp, Cavia porcellus
gcaacttccccactgcgcatcccctggctcctggtggctcctgggcggctcccttcacgcctggactccaggctctgccctgcgccgaggagcgcgcgccatggccccgcgaggaagcgcctggctgctgctgctgccggtggccctgctgctcgacgccgccacggcccaaggtgccagtcagttccgaatgtcaccccgtgaactggtcgcgcaagtcggcaccaaagtgaccctgcgctgtgaggtgctggtgcctaacgcgccggcgggatgctcgtggctcttccagccccgccacgacgccaaaggtcccaccttcctcctgtaccattcggcgtccgggaccaagttggccccagggctggaacagaagcgattcagcccctcgaagagcagtaacacctacaccctcacggtgaacagcttccagaagcgagacgaaggctactacttctgctcggtctccggcaacatgatgctctacttcagcccgttcgttcccgtcttcctgccagctcctcgcaccacgacgccccctccccctcccaccacgccgacccccagcgtgcagcccacgtcggtgcgccccgagacgtgtgtggtctctaagggcgcagcaggtgcgaggtggctggatctctcctgtgatgtctacatctgggcgcccctggccagcacatgcgcggcccttctgctggcactggtcatcacgatcatctgccaccgcaggaacagacaacgcgtttgcaaatgtcctaggccccaagccaggtctggaggcaaacccagcccttcagggaagttagtctaacaacatggcgcccagcctgtgcgaagccactacatgactttatactgagatcattccttggacagcaagtgctcctcttttgggtttcccagtcttccttcctatgtatttgttctcattactattttagtgggcatggggtgggaagagttgctttttcgttagacaaaaaataaaaccatgtagcatctgcagctcacaagggtcacagggctgttacctcacacaggggttagggtagcaagcagggctctcaggtactggaattcactcccttccactcacttgagggtgggcagcacccacgggtcatttatccctcatcatgctcctccacccacttgagctcagatgccacccaaagagcagtctatctaaacccaggccaaacacatgcaactgctttttgaacccgagagcctaatttatctgcagagaatgcaagtgctcctttgtcacttatatcttgtccatgacctttaataaatgtgctgcttttccctcaaaaaaaaaaaa

SEQ ID NO: 17 - protein, 242 aa, Bos taurus
MASLLTALILPLALLLLDAAKVLGSLSFRMSPTQKETRLGEKVELQCELLQSGMATGCSWLRHIPGDDPRPTFLMYLSAQRVKLAEGLDPRHISGAKVSGTKFQLTLSSFLQEDQGYYFCSVVSNSILYFSNFVPVFLPAKPATTPAMRPSSAAPTSAPQTRSVSPRSEVCRTSAGSAVDTSRLDFACNIYIWAPLVGTCGVLLLSLVITGICYRRNRRRVCKCPRPVVRQGGKPNLSEKYV

SEQ ID NO: 18 - DNA, 2001 bp, Bos taurus
gaattcggatccaccatggcctcactcttgaccgccctgatcctgccgctggccctgctgctgctcgatgccgccaaggtcctcgggtcgctctcgttccggatgtcgccgacgcagaaggagaccagactgggcgagaaggtggagctgcaatgcgagttgctgcagtccggcatggcgacagggtgctcctggctccgccacatacccggggacgaccccagacccaccttcctaatgtacctctccgcccaacgggtcaagctagccgagggactggaccccagacacatttccggcgccaaggtctccggcaccaaattccagctcaccctgagcagcttcctccaggaggaccaaggctactatttttgctcggtcgtgagcaactcgatactgtacttcagtaacttcgtgcctgtcttcttgccagcgaagccggccaccacgccggcgatgcggccatccagcgcggcgcccaccagcgcgccgcagactaggtcggtctctccgcgatcagaggtgtgccggacctcggcgggcagcgcagtggacacgagccggctggacttcgcctgcaatatctacatctgggctcccttggtcgggacctgcggcgtccttctcctgtcattggtcatcacaggcatctgctaccgccggaaccgaagacgtgtctgcaaatgtcccaggcctgtggtccgacaaggaggcaagcccaacctttcagagaaatatgtctaacatggcgatgggccccgtgtgacagccactacaagacttcgcactgagaactctcctgagatccttcccttttgatttctccctgcttccttccttctcgttattattatttttcatgggggtggggtgggaagagttactttttctttattatttactttgatacaaaacaagacactcgtgtctaaggcataccacaagggttatcatgctgttgtgctcccatactcgggtagagggcgggcgggccagagctaccgcaagctctattctcagaacctggctgtgagaactggtgggggcctcggcacccactcagccccaacttctcctccacccattttacaaaagaggacgctgaggcccagagatggggaacagctggatcagagtcccagcagggctccacacaactgagatctttcttctggaggcctctgtctcagcgtggggagctggatctcaagcctcagagaactagttatttctgaagcatctgtgatagacccatgactgcacccagagcctcgatgaggtaatgaaataggacaagaaaacttgacagagttctgtgatactgctgaacaggatcagattattttttttataatcaagcatgaaatgatacagataataggaattcttccaatgaagtggaaggagtgaactgaatgatggaaaatgagcaacctgacctctgaagaaaatctctgggaaatcccagcctggagatggttctcccagcccttgtattgcagaaggaccctcaaagaggagaggccaccctctgcaagcatgatttgagcgttaggaaagttgaatggagttcaagtctctctaaacattgagattccgtattcaaacatgctcctgggttatcggtgagtttttatagtttgtaaagggagaattgtgaccgagcagctggcacaggccctggcaccccaggctagcagctgagggaatgtgcagacactggtgaggaggctacgagcccagctgcagccctacaaggcatttccttccttactgtgttctgcaaaaaatgcatgctcactgggagaaaaaatgtagctaaggtagtaagaatcatccgtaattctttacctcagggataatccattgttaatattatgggctacattcttcctgattattttctgtgccctacatataaaatatataatttttaaaaatgggattgcactatgcttttataaatggctttaataaacaaacatttatggcttacttctt

SEQ ID NO: 19 - protein, 236 aa, Sus scrofa
MASLVTALLLPLVLQLHPAKVLGSSLFRTSPEMVQASLGETVKLRCEVMHSNTLTSCSWLYQKPGAASKPIFLMYLSKTRNKTAEGLDTRYISGYKANDNFYLILHRFREEDQGYYFCSFLSNSVLYFSNFMSVFLPAKPTKTPTTPPPKRTPTKASHAVSVAPEVCRPSGNADPRKLDLACDLYNWAPLVGTSGILLLSLVITIICHRRNRRRVCKCPRPVVRQGGKASPSERFI

SEQ ID NO: 20 - DNA, 2179 bp, Sus scrofa
atatcagcaaggcttgaggtgacatcacatcctccgaacgagaaaccgagaaaccgggctcggtggccggccgaagggcgcaacttcccccgtcgacgtcctactagctcttgcgcgcctccaggcttcgagcttccagcggagccgcgccgcggggagcgcgccatggcctcgctggtgaccgctctgctcctgccgctggtcctgcagctccatcccgccaaggtcctcgggtccagcttgttccggacgtcgccggagatggtgcaggctagcctgggagagacggtgaagctccgctgcgaggtgatgcactccaacacactgacaagctgttcctggctctaccagaagccgggggctgcctccaagcccatcttcctcatgtacctctccaaaacccggaataagacagccgaggggctggacacccgttacatctctggttacaaggccaatgacaacttctacctcatcctgcaccgcttccgcgaggaggaccaaggctactatttctgctcgttcctgagcaactcggttttgtatttcagcaacttcatgtccgtcttcttgccagcaaagcccaccaagacgccgactacgccaccacccaagcggactcccaccaaagcgtcgcacgccgtgtctgtggccccagaggtgtgccggccttcgggcaacgcagacccgaggaagctggacctcgcctgtgatctgtacaactgggcgcccctggttgggacctccggcatccttctcctgtcactggtcatcaccatcatctgccaccgccggaacagaagacgtgtttgcaaatgtcccaggcccgtggtcagacagggaggcaaggccagcccttcagagagattcatctaacatggcgacatgccccacgcagcagccactacaagacctcaaactgagacctctccgggcaggagagcaagggtcctttcctttccgtttccccagccttccttccttccttaagtattcttctcattattattatttccatgggggtggggtgggaagggtgactttttctttgggtgtttactttaattgacacaaaacgagactctatcacgtctttggtacgccgcaggggttcgaacaccgttgtgctcacacacacaacggtgaagggtgggcgggccagagctaccgcaagctgtgttctcagaaccaggctgtgagagctggtggggggtggggaggccctcggcacccacacaggccaaacctctccccctgccccccattttacaaaggaatgaggctgaggcccagagatggggggtggctggatcagagccccagcaaggctccaggctcatcctccacagcatttgggcctctcttccaggggcctctgtctcagctgggggagctgtgtctcccacctcaaggaaacaaggtttgcttgggcacctgtgatagactctgcactgtgcccagagccccggggaggcaatgcagtaagtcaaggggacgtgacagaggtctacggtgcagttgaacaggatcagatatattttttttaataatccagcatgaagttatatagataacaggaattcctcaaatagagtggaagggctgaactgaatcctggaaagtgaacaacacgacctctaaaggaaatccaatgcaaaaaatctctaagtggagacacagtggctctcccaggggacccatgaaagaggggaagccgccctttgcaaatatgatttgagcatcgcgaaagtcgaacggaggtcggccctctctaaatgtgagatctgatatttgaacgtgctcctcggatcattgatgggtttttttggtttgtaaacacagaattatgaccgagtagctggcctcccctggaccagcagctgtggatatggggcagactctgatgaggaggctaggagcccagactgctgccctctacgcgcatttcctctcttaaccatgttgtacaagaaatgcgtgctcgctggaagaaaaaactaaataataagagtcacccataattctttacttctggtataactcattgttaatattatggtgtacattcttcctgattattttctatgcacgtatataaaatgtatactttttaaaaatggaattgtactatgcttttagaagtggttttaataaacatttctgctatgaaaaaaaaaaa

SEQ ID NO: 21 - protein, 239 aa, Felis catus
MASPVTAQLLPLALLLHAAAAAGPSPFRLSPVRVEGRLGQRVELQCEVLLSSAAPGCTWLFQKNEPAARPIFLAYLSRSRTKLAEELDPKQISGQRIQDTLYSLTLHRFRKEEEGYYFCSVVSNSVLYFSAFVPVFLPVKPTTTPAPRPPTQAPITTSQRVSLRPGTCQPSAGSTVEASGLDLSCDIYIWAPLAGTCAFLLLSLVITVICNHRNRRRVCKCPRPVVRAGGKPSPSERYV

SEQ ID NO: 22 - DNA, 785 bp, Felis catus
atggcctctccggtgactgcccagctcctgccgctggccttgctgcttcatgccgccgcagccgccgggccgagcccgttccgcttatcgcccgtgagggtggagggcaggctcggccagcgggtggagctgcagtgcgaggtgctgctgtccagcgcggcgccgggctgcacctggctcttccagaagaacgaacctgccgcccgccccatcttcctggcgtacctctccagaagccggaccaagttggccgaggagctggaccccaaacagatctcgggccagaggattcaggacaccctctacagtctcaccctgcacagattccgcaaggaggaagaaggctactatttctgctcggtcgtgagcaactccgttctgtacttcagcgccttcgtcccggtcttcctgccagtcaagcccaccactacgcccgcgccgcgaccgcccacgcaggcgcccatcaccacgtcgcagcgggtgtctctgcgcccggggacctgccagccttcagcgggcagcacagtggaagcaagtgggctggatttgtcctgtgacatctacatctgggcacccctggctgggacctgcgccttccttctcctgtcgctggtcatcaccgtcatctgcaaccacaggaaccgaagacgtgtttgcaaatgtccgaggcccgtggtcagagcaggaggcaagcctagcccgtcagagagatacgtctaacatggagatgggccccatgcaccagccactacaagaccaaataaaactctctttatgaggacagt

SEQ ID NO: 23 - protein, 235 aa, Sigmodon hispidus
MAPRVTRFLCLTLLLEFIAELGGSKDFEMSPKKVVAHLGKEVRLTCEVWVSTSQGCSWLFLEHGSGVKPTFLIYLSGSRNERNNKIPSTKLSGKKEDKKYTLTLNNFAKEDEGYYFCSVTSNSVVYFSPLVSVFLPEKPTTPVPKPPTSVPTTAISRSLRPEACRPGAGTSVEKKGWDFDCDIIILAPLAGLCGVLLLSLVTTLICCHRNRKRVCKCPRPVVRQGGKPSPSGKLV

SEQ ID NO: 24 - DNA, 1229 bp, Sigmodon hispidus
ctcctgcttgacctaagctgctggtggaagcactgccatggccccccgggtgacccgctttctgtgcctgaccctgctgctggaatttatcgctgagctcggaggctcgaaagatttcgaaatgtctcctaagaaggtggtcgcccaccttggcaaggaggtgaggctaacatgcgaagtgtgggtgtctacttcgcaaggatgctcttggctcttcctggagcatggctccggagttaaacccactttcctcatctatctctctgggagccgcaacgaacggaataacaaaataccttcaactaagctatctgggaagaaggaagacaaaaagtacaccctcaccctgaataattttgctaaggaagacgaaggctactatttctgctctgtcacaagcaactcggtggtgtacttcagtcctctcgtgtcggtctttctgccagagaaacctaccacaccagtgccgaaaccacccacatcagtgcccactacggcgatatctcggtccctgcgaccagaagcttgccgacctggagccggcacctcagtggagaagaagggatgggacttcgactgtgatatcatcattttggcacccttagctggactctgtggggtccttctgctgtctctggtcaccacactcatctgctgccacaggaacagaaaacgagtctgcaaatgtcccaggcccgtggtcagacaaggaggcaagcccagcccttcagggaaactcgtgtaagatggcgccaagaaactacaactactacttcagagacctcttcatctagagctccagctctccttcttcaatttttctcaccttcctatatattgttctttgtattattttagtgggggtaggacagggttggaaccatttcctttctttatgaattcactttgacacaaaacaagaccacataatgtccacgggataccataagggcaggagctgttgctgcgtacatagcatgtgggggaagtacagaacagctgtctgggttctcaggatcagtggatgatcagcacccacttgatgatctaaatgccctgtctgcccattatatagaagaggttgaaggtcagaaatggggtgggcaggatctgtgcaccaggagagaacccaagctgacgaaatcctcactggatggctcagggaacttgcctctatatcctgagttctctttattcaggcctgtgcctggtagtgtgtaggctgagta

SEQ ID NO: 25 - protein, 235 aa, Saimiri sciureus
MASPVTALLLPLALLLHAARPSRFRVSPLDRTWNLGDKVELKCEVLLSNPSSGCSWLFQKRGAAASPTFLLYISQTKPKVADGLDAQRFSGKKMGDSFILTLRDFREEDQGFYFCSALSNSIMYFSPFVPVFLPAKPTTTPAPRPPTPEPTTASQPLSLRPQACRPPAGGAVDTRGLDFACDIYIWVPLAGTCGVLLLSLVITVYCNHRNRRRVCKCPRPAVKSGGKPSPSERYV

SEQ ID NO: 26 - DNA, 708 bp, Saimiri sciureus
atggcctctcccgtgaccgccttgctcctgccgctggccctgctgctccacgctgccaggccgagccggttccgggtgtcgccgctggatcggacctggaacttgggcgacaaggtggagctgaagtgcgaggtgctgctgtccaacccgtcctcgggctgctcgtggctcttccagaagcgcggcgctgccgccagccccaccttcctcctgtacatctcccaaaccaagcccaaggtggccgatgggctggacgcccagcgcttctccggcaagaagatgggggacagcttcattctcaccctgcgcgacttccgcgaggaggaccagggcttctatttctgctcggccctgagcaactccatcatgtacttcagccccttcgtgccggtcttcctgccagcgaagcccaccacgacgccagcgccgcgaccacccacaccggagcccaccaccgcgtcgcagcccctgtccctgcgtccacaggcttgccggcccccggcggggggcgcagtggacacgagggggctggacttcgcctgtgatatctacatctgggtgcccttggccgggacctgcggggtccttctcctgtcactggtcatcaccgtttattgcaatcacaggaaccgacgacgtgtttgcaaatgtccccggcctgcggtcaagtctggaggcaagcccagcccttcggagagatacgtctaa

SEQ ID NO: 27 - protein, 235 aa, Homo sapiens; identical to SEQ ID NO: 1

SEQ ID NO: 28 - DNA, 708 bp, Homo sapiens
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaa

SEQ ID NO: 29 - protein, 310 aa, Mus musculus
MASPLTRFLSLNLLLLGESIILGSGEAKPGAPELRIFPKKMDAELGGKVDLVCEVLGSVSGGCSWLFGNSSSKLPGPTFVVYMASSHNKITWDEKLNSSKLFSAMRDTNNKYVLTLNKFSKENEGYYFCSVISNSVMYFSSVVPVLGKVNSTTTKPVLRTPSPVHPTGTSGPGRPEDCRPRGSVKGTGLDFACDIYIWAPLAGICVALLLSLIITLICYHRSRKRVCKCPSIACLCLKLGGSKWYESVICSALAVSIRCNKSKSGELPLAVHLDIRAPCKNWEIAGSLVERYGKSGKHSPLSLKAVVESN

SEQ ID NO: 30 - DNA, 933 bp, Mus musculus; identical to SEQ ID NO: 8

SEQ ID NO: 31 - protein, 247 aa, Mus musculus; identical to SEQ ID NO: 11

SEQ ID NO: 32 - protein, 197 aa, Homo sapiens
MALPVTALLLPLALLLHAARPSQFRVSPLDRTWNLGWTVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAGNRRRVCKCPRPVVKSGDKPSLARYV

(19) United States
(12) Patent Application Publication
(10) Pub. No.: US 2005/0042217 A1
(43) Pub. Date: Feb. 24, 2005

(54) Specific inhibition of allorejection

(76) Inventors: Yan Qi, Highlands Ranch, CO (US); Xianghua Zhang, Aurora, CO (US); Paula J. Konigsberg, Denver, CO (US)

Correspondence Address: DORSEY & WHITNEY LLP;INTELLECTUAL PROPERTY DEPARTMENT, 4 EMBARCADERO CENTER, SUITE 3400, SAN FRANCISCO, CA, 94111, US

(21) Appl. No.: 10/804,762

(22) Filed: Mar. 19, 2004

Related U.S. Application Data
(60) Provisional application No. 60/456,378, filed on Mar. 19, 2003.

(51) Int. Cl. A61K 39/395

(52) U.S. Cl. 424/144.1

(57) ABSTRACT

The present invention provides methods and compositions for specifically inhibiting both cellular and humoral immune responses to alloantigen, thereby finding use in extending the survival of transplant allografts and treating graft versus host disease in transplant recipients.

### CROSS-REFERENCE TO RELATED APPLICATION(S)

This application claims the benefit of provisional application serial No. 60/456,378, filed Mar. 19, 2003.

### FIELD OF THE INVENTION

The present invention relates to immunosuppressive therapy, and more specifically, to methods and compositions for improving the outcome of transplantation procedures by specifically inhibiting the immune response to donor or host antigens in recipients of donor organs, tissues and cells.

### BACKGROUND OF THE INVENTION

The transplantation of allogeneic organs, tissues and cells has become increasingly important for the treatment of a wide variety of degenerative diseases and malignancies. The use of such allografts, however, is limited by graft rejection and/or graft versus host disease (GVHD) caused by antigenic differences between the donor and recipient, primarily involving antigens of the Major Histocompatibility Complex (“MHC”). Successful transplantation of non-lymphoid donor tissues and solid organs therefore depends on preventing the host immune response to donor antigen. Conversely, in transplants of donor lymphoid cells and tissue such as bone marrow transplantation (“BMT”) procedures, prevention of the donor immune response against host cells and tissues is necessary in order to avoid GVHD.

Priming of alloreactive T cells can occur through a direct allorecognition pathway facilitated by donor MHC molecules, as well as by an indirect allorecognition pathway wherein donor antigen is processed and presented in the context of self MHC by recipient antigen-presenting cells (APCs). Heeger, Am J. Transpl. 3: 525-533 (2003). Both CD4+ helper T cells as well as CD8+]cytotoxic T cells (CTLs) play important roles in the alloantigen immune response. Directly- and indirectly-primed CD4+T cells help in the production of alloantibody and provide the signals required for induction of CD8+ CTLs, both of which are capable of injuring the graft. Id. Given their central role in mediating both the cellular and humoral components of the immune response to alloantigens, some commentators have suggested that CD4+ helper T cells rather than CD8+ CTLs are essential for allorejection. Krieger et al., J. Exp. Med. 184:2013-2018 (1996). In any event, the success of any immunosuppressive strategy directed against the allorejection response depends on its ability to inhibit the activity of both subsets of T cells, and CD4+ T cells in particular.

Current therapeutic regimens use global immunosuppression of the host immune system in order to curtail the activities of both classes of T cells. Most regimens include a comprehensive induction phase at the time of transplantation, typically involving combinations of powerful T cell depleters such as anti-CD3 antibodies (e.g., OKT3) or anti-thymocyte globulins in conjunction with high-dose steroids, followed by the chronic administration of general immunosuppressive agents such as cyclosporin A (CsA), tacrolimus (FK-506), sirolimus (Rapamycin), azathioprine (Imuran®) and mycophenylate mofetil (CellCept®) for the life of the recipient. By permanently suppressing the recipient's general immune response, survival of the graft can be enhanced.

The global nature of this general immunosuppressive approach requires a constant balancing act by recipients and their clinicians throughout life, in order to achieve enough immune suppression to prevent graft rejection but not so much that the recipient's health is severely affected by adventitious infections. Unfortunately, even the right balance of immune suppression increases the risk of infection for the recipient, and a confluence of viral infection and permanent T cell suppression can lead to numerous hematologic malignancies unique to transplant recipients, such as post-transplant lymphoproliferative disorder (PTLD). Clearly, more specific immunosuppressive strategies that obviate the need for global suppression of the recipient's immune system are greatly needed.

The chronic nature of the current therapeutic approach is another major problem, both economically and physiologically. The yearly cost for maintenance immunosuppressive therapy can run as much as $10,000. More importantly, all of the current immunosuppressive drugs utilized for maintenance therapy have significant deleterious side effects that complicate their long-term use. For example, the two main drugs currently utilized for maintenance immunosuppression, CsA and FK506, are associated with significant nephrotoxicity over time. Paradoxically, then, long-term immunosuppression of a transplant recipient in some instances leads to the necessity of an additional organ transplant years later due to destruction of the recipient's kidneys resulting from maintenance therapy. Clearly, immunosuppressive strategies that reduce or better yet eliminate the need for chronic administration of these types of general immunosuppressive agents are also greatly needed.

Thus, a major unmet need in transplant immunobiology is the development of more specific immune inhibition strategies directed exclusively to the alloantigen response. Ideally, such strategies would inhibit allorejection without the need for continuous general pharmacologic immunosuppression and its attendant complications and costs. Achieving specific immunological tolerance to such alloantigens can improve transplant longevity and quality of life for the recipient, and at the same time considerably improve the cost effectiveness of transplant therapy. The present invention achieves these heretofore elusive goals.

### SUMMARY OF THE RELEVANT LITERATURE

It is known that the activity of MHC class I-restricted T cells (e.g., CD8+ CTLs) can be suppressed when a CTL that has received a signal through its T cell receptor complex also receives a signal through the α3 domain of its class I MHC molecule. This so-called veto signal may be delivered by a CD8 molecule expressed by the stimulator or “veto” cell. Sambhara and Miller, Science 252:1424-1427 (1991). The resulting immune suppression is both antigen-specific and MHC-restricted, and results from the unidirectional recognition of the veto cell by the responding CTL, but not vice versa. Rammensee et al., Eur. J. Immunol. 12:930-934 (1982); Fink et al., J. Exp. Med. 157:141-154 (1983); Rammensee et al., J. Immunol. 132:668-672 (1984). Veto activity has since been linked to the presence of the CD8 α chains, such that the veto function is lost if expression of CD8 is deleted and established when the CD8 α chain is expressed. Hambor et al., J. Immunol. 145:1646-1652 (1990); Hambor et al., Intern. Immunol. 2:8856-8879 (1990); Kaplan et al., Proc. Natl. Acad. Sci. USA 86:8512-8515 (1989).

Numerous strategies have been proposed to exploit this antigen-specific suppressive pathway to eliminate unwanted cytotoxic T cell responses. One such strategy involves the use of polypeptide conjugates covalently linking CD8 or a functional domain thereof to secondary ligands that direct CD8's veto activity to specific target cells. See, e.g. U.S. Pat. Nos. 5,242,687, 5,601,828 and 5,623,056. Alternatively, hybrid antibody molecules have been investigated having a monoclonal antibody binding site with specificity to MHC class I molecules linked to the extracellular domain of the CD8 α chain. Qi et al., J. Exp. Med. 183:1973-1980 (1996). Such molecules, however, have several shortcomings and have yet to find actual clinical utility.

More recently, International PCT Publication No. WO 02/102852 describes the inhibition of CTL using soluble C8 α-chain variants having amino acid modifications designed to increased affinity for MHC class I. Significantly, and consistent with the above prior art, it is taught therein that the proposed CD8α compositions are specific for class I MHC molecules and are therefore expected to inhibit only the response of CD8+ CTL. Id. at p. 27. It is further suggested that combinations with other immunosuppressive agents will be required in situations involving other elements of the cellular and humoral immune responses, e.g., MHC class II-restricted T cells such as CD4+ T cells. Id. p. 28.

### SUMMARY OF THE INVENTION

The present invention is based on the surprising discovery that the veto effect mediated by targeted expression of CD8α can effectively and specifically suppress responding CD4+ T cells (MHC class II-restricted) as well as CD8+T cells (MHC class I-restricted), and the resulting determination that both the cellular and humoral components of the immune response directed against alloantigens can therefore be inhibited. Thus, using the compositions and methods described herein one may selectively inhibit allorejection activity directed to either donor or host antigens without the need for chronic administration of general immunosuppressive agents, effectively resulting in specific immunological tolerance to donor tissue, organs and/or cells.

In accordance with the invention, methods and compositions are provided for specifically inhibiting an alloantigen response to donor and/or host antigens, depending on the nature of the allograft, in order to prolong the survival of allogeneic grafts and protect the health of the transplant recipient. Preferably, the subject compositions and methods are capable of inhibiting both the humoral and cellular immune responses to such alloantigens. Still more preferably, the subject compositions and methods are capable of inducing stable and specific immunological tolerance to such alloantigens without the need for chronic immunosuppressive therapy.

In one aspect, methods for specifically inhibiting immune responses to alloantigens are provided, comprising contacting a target cell expressing at least one such alloantigen with an expression vector encoding all or a functional portion of a CD8 polypeptide, preferably a human CD8 polypeptide, still more preferably the human CD8 a-chain, whereby the CD8 polypeptide is expressed by the target cell and whereby the alloimmune response directed against the alloantigen is specifically inhibited. In one embodiment, the alloantigen comprises a donor alloantigen and the target cell comprises an allograft cell. In an alternative embodiment, the alloantigen comprises a recipient alloantigen and the target cell comprises a recipient cell. In a further embodiment, the alloimmune response includes both a humoral component and a cellular component. In a preferred embodiment, the alloimmune response is effectively inhibited without the need for general immunosuppressive agents.

In another aspect, methods for specifically inhibiting immune responses to donor alloantigen are provided, comprising conditioning donor allograft cells in vivo or ex vivo to express all or a functional portion of a CD8 polypeptide, preferably a human CD8 polypeptide, still more preferably the human CD8 α-chain. In one embodiment, the conditioning step comprises contacting the allograft cells in vivo or ex vivo with an expression vector encoding all or a functional portion of a CD8 polypeptide, whereby the CD8 polypeptide is expressed by allograft cells and whereby the recipient immune response directed against donor alloantigen is specifically inhibited. Preferably, both the cellular and humoral components of the recipient alloimmune response are effectively and specifically inhibited without the need for general immunosuppressive agents.

In another aspect, methods for specifically inhibiting immune responses to recipient alloantigen are provided, comprising in vivo conditioning of recipient cells to express all or a functional portion of a CD8 polypeptide, preferably a human CD8 polypeptide, still more preferably the human CD8 α-chain. Preferred recipient cells for the subject conditioning step include those found in the recipient tissues and organs most at risk of a GVHD immune response such as, e.g., liver, skin and intestinal tract. In one embodiment, the conditioning step comprises contacting such recipient cells in vivo with an expression vector encoding all or a functional portion of a CD8 polypeptide, whereby the CD8 polypeptide is expressed by the cells and whereby the donor immune response directed against recipient alloantigen is specifically inhibited. Preferably, the donor alloimmune response is effectively and specifically inhibited without the need for general immunosuppressive agents.

Also provided are methods for prolonging the survival of an allograft in a recipient, comprising conditioning the allograft cells in vivo or ex vivo to express all or a functional portion of a CD8 polypeptide, preferably a human CD8 polypeptide, still more preferably the human CD8 α-chain. In one embodiment, the conditioning step comprises contacting the allograft cells in vivo or ex vivo with an expression vector encoding all or a functional portion of a CD8 polypeptide, wherein the CD8 polypeptide is expressed by allograft cells and whereby the survival time of the allograft in the recipient is extended. Preferably, the conditioning step is performed prior to or contemporaneously with transplantation of the allograft. Still more preferably, the conditioning step is performed ex vivo prior to transplantation of the allograft, or in vivo in the donor prior to or contemporaneous with harvesting of the allograft. Most preferably, use of the subject methods is effective to induce stable immunological tolerance to the allograft, such that chronic administration of general immunosuppressive agents will not be required.

Also provided are methods for suppressing GVHD in a recipient, comprising in vivo conditioning of recipient cells at risk of a GVHD immune response to express all or a functional portion of a CD8 polypeptide, preferably a human CD8 polypeptide, still more preferably the human CD8 α-chain. In one embodiment, the conditioning step comprises contacting recipient cells in vivo with an expression vector encoding all or a functional portion of a CD8 polypeptide, whereby the CD8 polypeptide is expressed by the cells and whereby the GVHD immune response raised against the recipient cells by transplanted donor T cells is suppressed. Preferably, the conditioning step is performed contemporaneously with or subsequent to transplantation of the allograft. Still more preferably, the conditioning step is performed in vivo in the recipient after transplantation of the allograft. Most preferably, use of the subject methods is effective to induce stable immunological tolerance of transplanted donor T cells to recipient alloantigen, such that chronic administration of general immunosuppressive agents is not needed.

Preferred CD8 polypeptides for use in the subject methods and compositions will generally comprise the CD8 α-chain, more preferably the extracellular domain of the CD8 α-chain, and still more preferably the Ig-like domain of the CD8 α-chain. In alternative preferred embodiments, the CD8 polypeptides may comprise or consist essentially of the extracellular domain of the CD8 α-chain and a transmembrane domain, or more preferably the Ig-like domain of the CD8 α-chain and a transmembrane domain. In a particularly preferred embodiment, the transmembrane domain is the transmembrane domain of the CD8 α-chain. Given the nature of the subject expression methods, as well as the apparent inadequacies of the prior art soluble forms of CD8 α-chain described above, the presence of the CD8 α-chain transmembrane domain or a suitable alternative transmembrane region is deemed essential.

Suitable expression vectors contemplated for use in the subject methods and compositions include recombinant and non-recombinant vectors, and viral vectors (e.g., adenoviral, retroviral, adeno-associated viral vectors and the like) as well as non-viral vectors (e.g., bacterial plasmids, phages, liposomes and the like) vectors.

In a further aspect, the present invention provides improved transplant allografts capable of specifically and effectively inhibiting a recipient immune response raised against them. In one embodiment, the improved transplant allograft comprises allograft cells modified to express a CD8 polypeptide, preferably a human CD8 polypeptide, still more preferably the human CD8 α-chain. As discussed herein, the CD8 polypeptide may comprise or alternatively consist essentially of the extracellular domain of the CD8 α-chain and a transmembrane domain, or the Ig-like domain of the CD8 α-chain and a transmembrane domain. The transmembrane domain may be that of the CD8 α-chain or may be another advantageously-selected transmembrane domain. Modification of allograft cells may be achieved with a liposome-mediated nucleic acid transfer vehicle, a viral-mediated nucleic acid transfer vehicle, and the like, as disclosed herein.

In a still further aspect, an improved organ preservation solution is provided, comprising an expression vector encoding a CD8 polypeptide. Thus, in a preferred embodiment, the invention provides an improved organ preservation solution comprising an expression vector comprising a nucleic acid encoding for a CD8 polypeptide, preferably a human CD8 polypeptide, and most preferably the human CD8 α-chain. In another preferred embodiment, the improved organ preservation solution comprises an expression vector comprising a nucleic acid encoding for the extracellular domain of a CD8 α-chain and a transmembrane domain, or alternatively the Ig-like domain of the CD8 α-chain and a transmembrane domain. In a particularly preferred embodiment, the transmembrane domain is the CD8 α-chain transmembrane domain. In further embodiments, the vector may further comprise a nucleic acid encoding for an anti-inflammatory molecule such as, e.g., heme oxygenase.

In a broader aspect, methods are provided for specifically inhibiting a host immune response to a target cell-specific antigen, comprising conditioning the target cell in vivo or ex vivo to express all or a functional portion of a CD8 polypeptide, more preferably the human CD8 polypeptide, still more preferably the human CD8 α-chain, wherein the CD8 polypeptide is expressed by the target cell and whereby an immune response directed against such antigen is specifically inhibited. In one embodiment, the target cell-specific antigen is an alloantigen. In another embodiment, the target cell-specific antigen is an autoantigen. In a preferred embodiment, the conditioning step comprises contacting the target cell in vivo or ex vivo with an expression vector encoding the CD8 polypeptide.

In a still further aspect, methods for preventing the development of and for treating autoimmune diseases are provided, comprising administering to a patient in need thereof a therapeutic composition comprising an expression vector encoding all or a functional portion of a CD8 polypeptide, preferably a human CD8 polypeptide, still more preferably the CD8 α-chain, wherein expression of the CD8 polypeptide by a contacted target cell specifically inhibits an autoreactive immune response directed against the target-cell specific autoantigens.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 depicts CD8 α-chain protein and nucleic acid sequences from various species. Also included are accession numbers for the noted sequences.

FIGS. 2A-B depict the amino acid and nucleic acid sequences for the wild-type human CD8 α-chain, including a demarcation of the different domains of the protein for human and mouse, respectively.

FIG. 3 depicts Balb/c spleen cells that were stimulated with C57BL/6 spleen cells. Cultures were supplemented with normal fibroblasts, medium or fibroblasts with CD8 of mouse (A) or human (B) origin. Cultures were harvested and tested for their lytic ability towards C57BL/6-derived target cells.

FIG. 4 depicts Balb/c (H-2d) mice that were injected with control fibroblasts (▪ and ▴) or mCD8-transfected C57BL/6-(H-2^{b}) derived (O and ●) fibroblasts. After two weeks animals were sacrificed, spleen cells were harvested, stimulated with C57BL/6 (H-2^{b}) (▪and O) or CBA/J (H-2^{k}) (● and ▴) spleen cells and tested for their lytic ability on EL4 (H-2b) (▪ and O) or S.AKR (H-2^{k}) (● and ▴) target cells.

FIG. 5 depicts target cells (▴) or CD8-expressing targets (▪) that were tested for their susceptibility to lysis by alloreactive T cells (A) or by antigen-specific CTLs (B).

FIG. 6 depicts MLCs (Balb/c anti-C57B/6) that were set up in the presence of normal fibroblasts (●) and fibroblasts transduced with mAdCD8 (A, ▴) or HAdCD8.(B, ▴). No fibroblasts were added to control cultures (▪). The lytic activity of these cultures towards an C57BL/6-derived target was determined at the end of the culture period.

FIG. 7 depicts immunization with an adenoviral veto transfer vector, mAdCD8. C57BL/6 mice were infected with the vectors indicated above. After 10 days, spleen cells were harvested and cultured in the presence of the Adβgal virus. The number of blast cells is given.

FIG. 8 depicts negative immunization with mAdCD8 (A) C57BL/6 mice were once immunized i.v. with Adβgal or mAdCD8. (B) Animals treated as in (A) were re-immunized with Adβgal after 5 days. Seven days after the last injection animals were sacrificed, and their spleen cells were cultured in the presence of Adβgal. After 5 days of culture, cells were tested for their lytic ability of Adβgal-infected syngeneic target cells.

FIG. 9 depicts 3×106 C7Bl/6 spleen cells that were incubated with 1×106 (or no) stimulator cells, transduced as indicated. After 4 days the cultures were analyzed for presence CD4+ T lymphoblasts by immunofluorescence.

FIGS. 10A-D depicts surface expression of mouse and human CD8 α-chains after infection with the different virus constructs. A. Infected cells: MC57T Fibroblasts; Panel 1: Mock-Infection; Panel 2: Infection with hAdCD8. B. Infected cells: MC57T Fibroblasts; Panel 1: Mock Infection; Panel 2: Infection with mAdCD8. C. Infected cells: Balbc unselected bone marrow cells; Panel 1: Infection with lacZ Adeniviral Vector (AdLacZ); Panel i2: Infection with mAdCD8. D. Infected Cells: MC57T Fibroblasts; Panel 1: Mock-Infection; Panel 2: Invection with pAAV-mCD8; Panel 3: Infection with pAAV-hCD8.

FIG. 11 depicts MLCs (Balb/c anti-C57BL/6) were set up in the presence of these fibroblasts that had been cultured for 0 or 5 hours after transduction before they were added to the MLCs. At the end of the cultures, the number of lymphoblasts was determined on a fluorescence activated cell analyzer.

FIG. 12 depicts in vitro inhibition with veto transfer vector. A Balb/c anti-C57BL/6 mixed lymphocyte culture (MLC) was established in the absence or presence of uninfected or mAdCD8-infected MC57 fibroblasts (H-2b) (X). CTL responses were measured in EL4 (H-2b) target cells.

FIG. 13 depicts Balb/c mice that were immunized with AdLacZ (Δ) or mAdCD8 (□). Their spleen cells were cultured in the presence of AdLacZ and tested for specific lytic activity against AdLacZ-infected syngeneic P815 target cells.

FIGS. 14A-B depicts (A) C57BL/6 animals that were immunized with AdLacZ (□) or mAdCD8 (Δ). The lytic activity of their spleen cells towards syngeneic AdLacZ EL4 target cells was tested. (B) Such animals were re-immunized with AdLacZ prior to testing their lytic activity against AdLaz-infected EL4 targets.

FIG. 15 depicts single cell suspensions that were prepared from newborn hearts. The heart muscle cells were transduced with mAdCD8 (B) or mock-infected, cultured for 48 hours and stained for the surface expression of CD8.

FIG. 16 depicts newborn C57BL/6 hearts that were infected with 109 (□), 5×107 (Δ), 107 (◯) PFU AdCD8 or mock-infected (◯). Thirty-five days after transplantation into BALB/c recipients, the activity of the lytic activity of activated recipient T cells was tested on donor-type target cells.

FIG. 17 depicts newborn C57BL/6 hearts that were infected with AdCD8 (□) or mock-infected (◯). Thirty-eight days after transplantation into Balb/c recipients, the activity of the lytic activity of activated recipient T cells was tested on donor-type target cells.

FIG. 18 depicts C57BL/6 hearts infected with mAdCD8 (treated) or mock-infected (control) were transplanted into Balb/c mice. After 52 days, the animals were sacrificed and the tissue was stained (HE) and the lytic activity of recipient T cells was tested on donor-type target cells.

FIG. 19 depicts pancreatic islet transplantation protocol.

FIG. 20 depicts blood glucose levels in normal (□) and Streptpzotocin-treated (◯) mice.

FIG. 21 depicts syngeneic pancreatic islet transplants performed in Balb/c (□) and in C57BL/6 (◯) mice.

FIG. 22 depicts transplantation of syngeneic mAdCD8-transduced pancreatic islets harvested from Balb/c (◯) or C57BL/6 (□) mice.

FIG. 23 depicts viability of transplanted islets. Blood sugar levels in Balb/c mice with chemically induced diabetes mellitus that had received a transplant of fully allogeneic mAdCD8 transduced C57Bl/6 pancreatic islets.

FIG. 24 depicts the suppression of transplant-specific CTLs in an assay designed to recognize allogeneic targets following lung transplant.

FIG. 25 depicts insulin production in mice transplanted with mAdCD8-transduced C57BL/6 pancreatic islets.

### DETAILED DESCRIPTION OF THE INVENTION

Alloimmune responses directed against donor and/or host antigens represent a continuing medical challenge to the success of transplantation procedures. As noted above, the success of the present invention stems from the surprising discovery that the modification of an allograft to express an immunomodulatory molecule such as CD8, and particularly the CD8 α-chain, will effectively and specifically inhibit both the humoral and the cellular components of the immune response directed against target cell-specific antigens.

In accordance therewith, the present invention provides compositions and methods for inhibiting and/or suppressing alloimmune responses directed against a target cell expressing an alloantigen, comprising conditioning the cell to express all or a functional portion of an immunomodulatory molecule, preferably a CD8 polypeptide, still more preferably the CD8 α-chain. In the preferred embodiment, the conditioning step comprises contacting the cell with an expression vector encoding for all or a functional portion of a CD8 polypeptide as described herein. The invention further contemplates alternative conditioning methods for modulating expression levels of CD8 in a target cell to effectively and specifically inhibit an immune response against the target cell such as, e.g., providing transcriptional activators that result in increased CD8 expression. See, e.g., Mortlock et al., Nuc. Acids. Res. 31:152 (2003); Mizuguchi et al., Hum. Gene Ther. 14:1265-77 (2003). The specificity and selectivity of the immune inhibition achieved by the subject methods and compositions provides a significant improvement over conventional immunosuppressive strategies, which typically produce a generalized and non-specific inhibition of the immune system thereby leaving the host highly susceptible to adventitious infections and, in some cases, mutagenicity and cancer.

The methods described herein can be used alone or in combination with other methods, such as the administration of other active agents, e.g., therapeutic or prophylactic agents and/or general immunosuppressive agents (e.g., cyclosporin, FK506), different antibodies etc. as are known in the art. Preferably, the use of such agents is unnecessary in view of the alloantigen-specific immunosuppression obtained using the subject compositions and methods.

By “inhibiting” is meant the direct or indirect, partial or complete, inhibition and/or reduction of an innate or acquired immune response, whether cellular (e.g., leukocyte recruitment) or humoral, to target cell-specific antigens. Target cell-specific antigens include any unique antigen associated with a target cell of interest including, e.g., alloantigens expressed by transplanted organs, tissue and cells (divergent HLA molecules, etc.) or self-antigens associated with an autoimmune disorder (autoantigens) including, e.g., myelin basic protein (MBP), proteolipid protein PLP-1, myelin oligodendrocyte glycoprotein, pro-insulin/insulin, glutamic acid decarboxylase (GAD), matrix metalloproteinase (MMP-1), type II collagen, thyroglobulin, and the like.

By “immune response” is preferably meant an acquired immune response, such as a cellular or humoral immune response.

By “specific immune inhibition” or “antigen-specific immune inhibition” is meant the inhibition of immune responses directed against antigens such as alloantigens, as opposed to general immune inhibition which is not antigen-specific. Thus, by way of example, the absence of a recipient cellular and/or humoral immune response to donor antigens, combined with evidence of in vivo immune competence to other foreign antigens, would demonstrate specific immune inhibition of donor alloantigen.

By “stable immunological tolerance” is meant stable, long-term allograft survival and/or function for at least one year without the use of general immunosuppressive agents.

By “expression vector” is meant any vehicle for delivery of a nucleic acid to a target cell. Expression vectors can be generally divided into viral vectors and non-viral vectors. By viral vectors is meant, but not limited to adenoviral vectors, adeno-associated vectors, retroviral vectors, lentiviral vectors, and the like. By non-viral vectors is meant plasmid vectors, naked DNA, naked DNA coupled to different carriers, or DNA associated with liposomes or other lipid preparation. Generally, expression vectors are recombinant, although in some embodiments, for example when liposomes or cell ablation, e.g. biolostic techniques, are used, they are not. Preferred recombinant vectors for use herein are plasmid vectors as well as viral vectors selected from the group consisting of an adenoviral vector, an adeno-associated viral vector, a herpes viral vector and a retroviral vector. In some embodiments utilizing recombinant viral vectors, and in particular adenoviral vectors, the immunogenicity of the capsid, e.g., the hexon protein of an adenoviral capsid, may be reduced in accordance with methods known in the art, although such modifications are no longer a necessity in view of the improvements detailed herein.

By “contacting” is meant administering the gene therapy expression vector to the cell in such a manner and in such an amount as to effect physical contact between the vector and cell. If the vector is a recombinant viral particle, desirably, attachment to and infection of the cell by the viral vector is effected by such physical contact. If the viral vector is other than a recombinant viral particle, such as a nonencapsulated viral nucleic acid or other nucleic acid, desirably, entry into the cell by the nucleic acid is effected.

Such “contacting” can be done by any means known to those skilled in the art, and described herein, by which the apparent touching or mutual tangency of the vector with the target cell can be effected. Optionally, the vector, such as an adenoviral vector, can be further complexed with a bispecific or multispecific molecule (e.g., an antibody or fragment thereof, in which case “contacting” involves the apparent touching or mutual tangency of the complex of the vector and the bispecific or multispecific molecule with the target cell. For example, the vector and the bispecific (multispecific) molecule can be covalently joined, e.g., by chemical means known to those skilled in the art, or other means. Preferably, the vector and the bispecific (multispecific) molecule can be linked by means of noncovalent interactions (e.g., ionic bonds, hydrogen bonds, Van der Waals forces, and/or nonpolar interactions). Although the vector and the bispecific (multispecific) molecule can be brought into contact by mixing in a small volume of the same solution, the target cell and the complex need not necessarily be brought into contact in a small volume, as, for instance, in cases where the complex is administered to a host (e.g., a human), and the complex travels by the bloodstream to the target cell to which it binds selectively and into which it enters. The contacting of the vector with a bispecific (multispecific) molecule preferably is done before the target cell is contacted with the complex of the vector and the bispecific (multispecific) molecule.

In further embodiments, the expression vector may optionally further include nucleic acid encoding an additional therapeutic molecule of interest such as, e.g., anti-inflammatory molecules such as heme oxygenase, along with the nucleic acid encoding for the self-antigen and the immunomodulatory CD8 polypeptide. Alternatively, separate expression vectors can be utilized in order to independently optimize timing of the presentation of the therapeutic molecule(s) and the CD8 polypeptide to the target cells. The beneficial effects of heme oxygenase expression on reducing ischemic/reperfusion injury are well documented. See, e.g., International Publication No. WO 00/36113, the disclosure of which is expressly incorporated by reference herein.

A “target cell” can be present as a single entity, or can be part of a larger collection of cells. Such a “larger collection of cells” may comprise, for instance, a cell culture (either mixed or pure), a tissue (e.g., epithelial or other tissue), an organ (e.g., heart, lung, liver, gallbladder, urinary bladder, eye or other organ), an organ system (e.g., circulatory system, respiratory system, gastrointestinal system, urinary system, nervous system, integumentary system or other organ system), or an organism (e.g., a bird, mammal, particularly a human, or the like). Preferably, the organs/tissues/cells being targeted are of the circulatory system (e.g., including, but not limited to heart, blood vessels, and blood), respiratory system (e.g., nose, pharynx, larynx, trachea, bronchi, bronchioles, lungs, and the like), gastrointestinal system (e.g., including mouth, pharynx, esophagus, stomach, intestines, salivary glands, pancreas, liver, gallbladder, and others), urinary system (e.g., such as kidneys, ureters, urinary bladder, urethra, and the like), nervous system (e.g., including, but not limited to, brain and spinal cord, and special sense organs, such as the eye) and integumentary system (e.g., skin). Even more preferably, the cells are selected from the group consisting of heart, blood vessel, lung, liver, gallbladder, urinary bladder, eye cells and stem cells. Methods of culturing and using stem cells are disclosed in more detail in U.S. Pat. Nos. 5,672,346, 6,143,292 and 6,534,052, which are incorporated herein by reference.

In particular, a target cell with which an expression vector such as a viral vector or plasmid is contacted differs from another cell in that the contacted target cell comprises a particular cell-surface binding site that can be targeted by the expression vector. By “particular cell-surface binding site” is meant any site (i.e., molecule or combination of molecules) present on the surface of a cell with which the vector, e.g., adenoviral vector, can interact in order to attach to the cell and, thereby, enter the cell. A particular cell-surface binding site, therefore, encompasses a cell-surface receptor and, preferably, is a protein (including a modified protein), a carbohydrate, a glycoprotein, a proteoglycan, a lipid, a mucin molecule or mucoprotein, and the like. Examples of potential cell-surface binding sites include, but are not limited to: heparin and chondroitin sulfate moieties found on glycosaminoglycans; sialic acid moieties found on mucins, glycoproteins, and gangliosides; major histocompatability complex I (MHC I) glycoproteins; common carbohydrate molecules found in membrane glycoproteins, including mannose, N-acetyl-galactosamine, N-acetyl-glucosamine, fucose, and galactose; glycoproteins, such as ICAM-1, VCAM, E-selectin, P-selectin, L-selectin, and integrin molecules; and tumor-specific antigens present on cancerous cells, such as, for instance, MUC-1 tumor-specific epitopes. However, targeting an expression vector such as an adenovirus to a cell is not limited to any specific mechanism of cellular interaction (i.e., interaction with a given cell-surface binding site).

As used herein and further defined below, “polynucleotide” or “nucleic acid” may refer to either DNA or RNA, or molecules which contain both deoxy- and ribonucleotides. The nucleic acids include genomic DNA, cDNA and oligonucleotides including sense and anti-sense nucleic acids. Such nucleic acids may also contain modifications in the ribose-phosphate backbone to increase stability and half life of such molecules in physiological environments.

The nucleic acid may be double stranded, single stranded, or contain portions of both double stranded or single stranded sequence. As will be appreciated by those in the art, the depiction of a single strand (“Watson”) also defines the sequence of the other strand (“Crick”); thus the sequences depicted in the Figures also include the complement of the sequence. By the term “recombinant nucleic acid” herein is meant nucleic acid, originally formed in vitro, in general, by the manipulation of nucleic acid by endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it may replicate non-recombinantly, i.e. using the in vivo cellular machinery of the host cell rather than in vitro or extrachromosomal manipulations; however, such nucleic acids, once produced recombinantly, although subsequently replicated non-recombinantly, are still considered recombinant for the purposes of the invention.

The terms “polypeptide” and ∫protein” may be used interchangeably throughout this application and mean at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradation. Alterations of native amino acid sequences to produce variant proteins and peptides for targeting or expression as a transgene, for example, can be done by a variety of means known to those skilled in the art. A variant peptide is a peptide that is substantially homologous to a given peptide, but which has an amino acid sequence that differs from that peptide. The degree of homology (i.e., percent identity) can be determined, for instance, by comparing sequence information using a computer program optimized for such comparison (e.g., using the GAP computer program, version 6.0 or a higher version, described by Devereux et al. (Nucleic Acids Res., 12, 387 (1984)), and freely available from the University of Wisconsin Genetics Computer Group (UWGCG)). The activity of the variant proteins and/or peptides can be assessed using other methods known to those skilled in the art.

In terms of amino acid residues that are not identical between the variant protein (peptide) and the reference protein (peptide), the variant proteins (peptides) preferably comprise conservative amino acid substitutions, i.e., such that a given amino acid is substituted by another amino acid of similar size, charge density, hydrophobicity/hydrophilicity, and/or configuration (e.g., Val for Phe). The variant site-specific mutations can be introduced by ligating into an expression vector a synthesized oligonucleotide comprising the modified site. Alternately, oligonucleotide-directed site-specific mutagenesis procedures can be used, such as those disclosed in Walder et al., Gene, 42:133 (1986); Bauer et al., Gene, 37:73 (1985); Craik, Biotechniques, January 1995, pp. 12-19; and U.S. Pat. Nos. 4,518,584 and 4,737,462.

Immunomodulatory Molecules

In the context of the present specification, an “immunomodulatory molecule” is an polypeptide molecule that modulates, i.e. increases or decreases a cellular and/or humoral host immune response directed to a target cell in an antigen-specific fashion, and preferably is one that decreases the host immune response. Generally, in accordance with the teachings of the present invention the immunomodulatory molecule(s) will be associated with the target cell surface membrane, e.g., inserted into the cell surface membrane or covalently or non-covalently bound thereto, after expression from the vectors described herein.

In preferred embodiments, the immunomodulatory molecule comprises all or a functional portion of a CD8 protein, and even more preferably all or a functional portion of the CD8 a chain. For human CD8 coding sequences, see Leahy, Faseb J. 9:17-25 (1995); Leahy et al., Cell 68:1145-62 (1992); Nakayama et al., Immunogenetics 30:393-7 (1989). By “functional portion” with respect to CD8 proteins and polypeptides is meant that portion of the CD8 α-chain retaining veto activity as described herein, more particularly that portion retaining the HLA-binding activity of the CD8 α-chain, and specifically the Ig-like domain in the extracellular region of the CD8 α-chain. Exemplary variant CD8 polypeptides are described in Gao and Jakobsen, Immunology Today 21:630-636 (2000), herein incorporated by reference. In some embodiments, the full length CD8 α-chain is used. However, in some embodiments the cytoplasmic domain is deleted. Preferably the transmembrane domain and extracellular domain are retained.

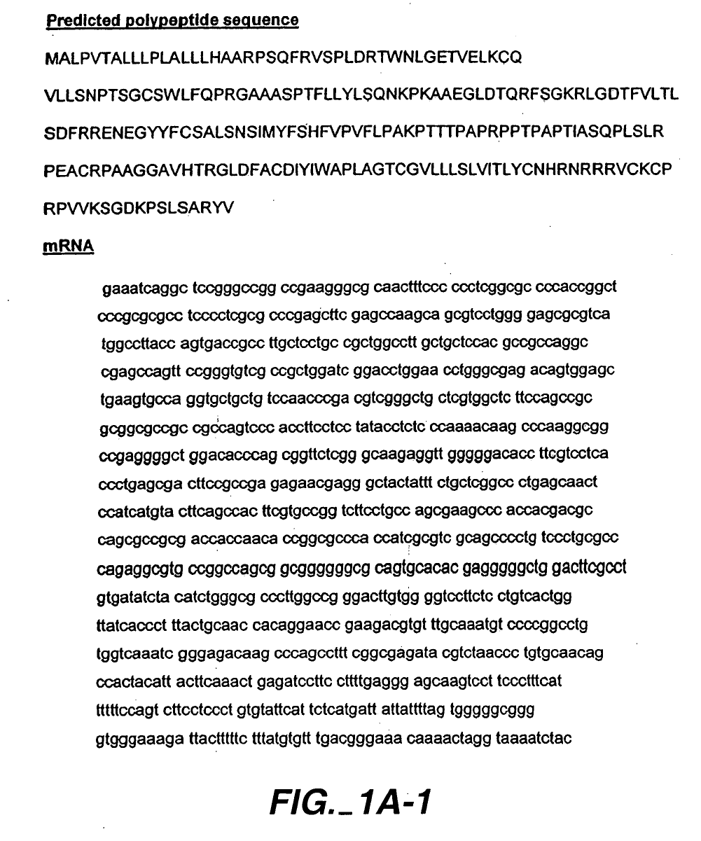

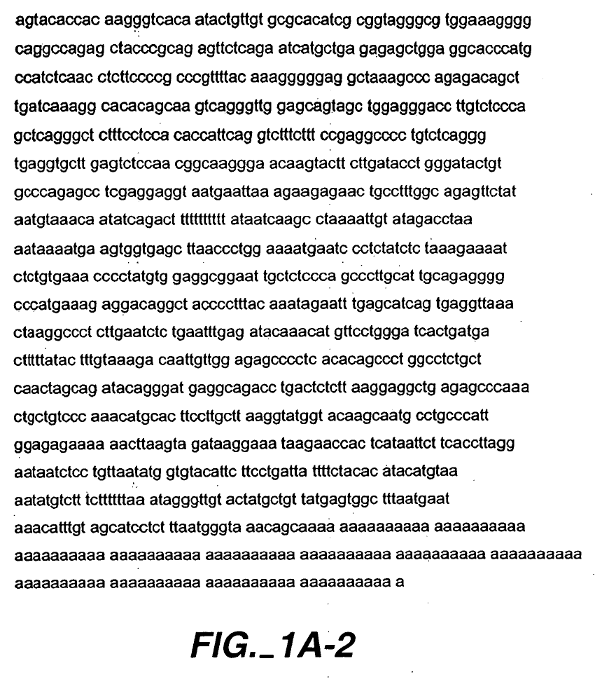

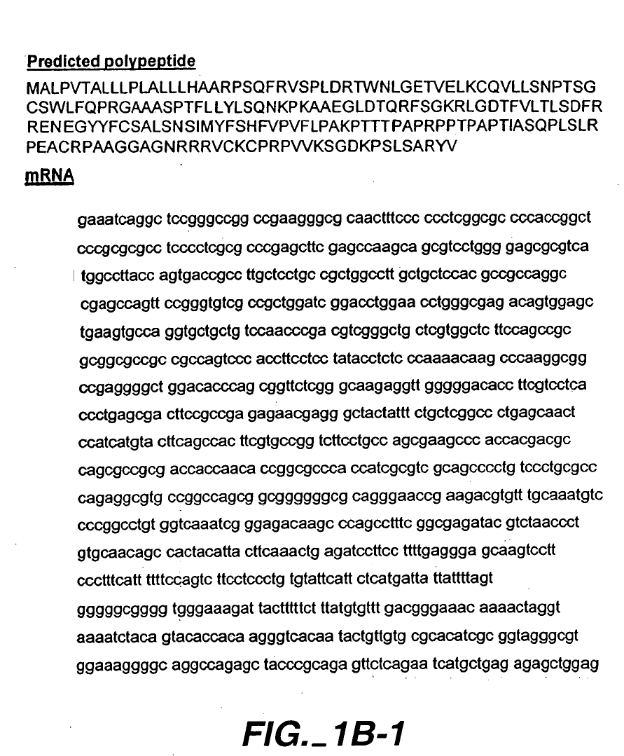

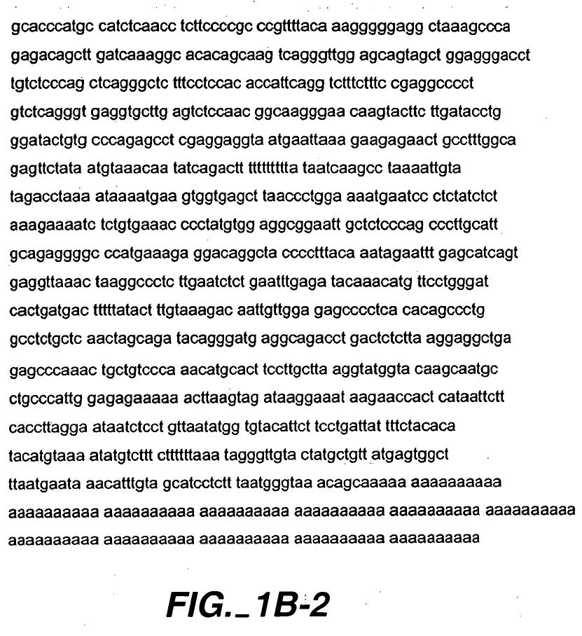

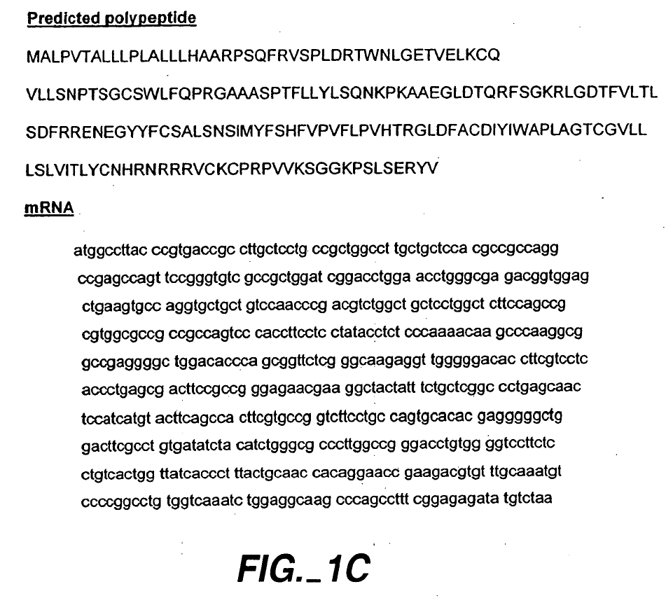

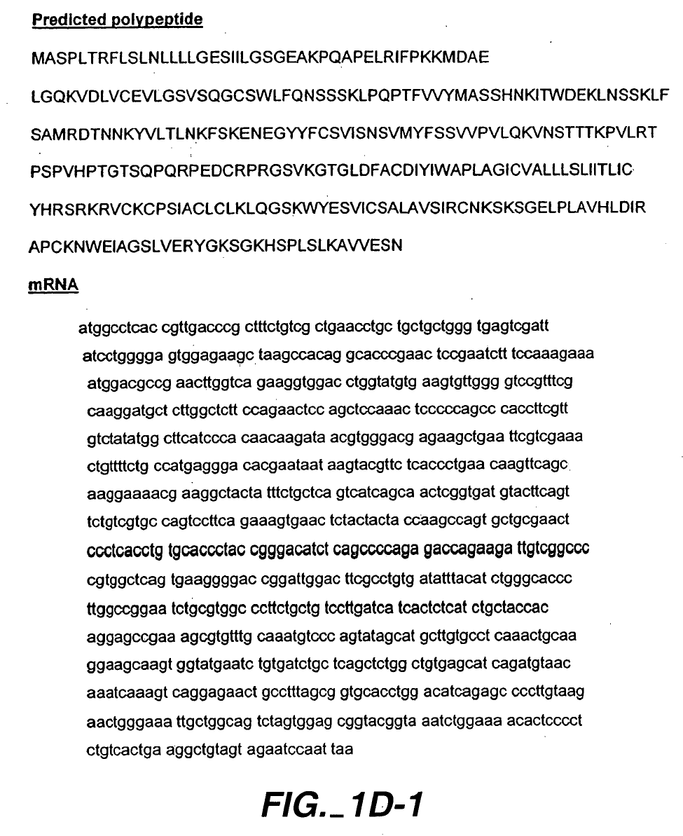

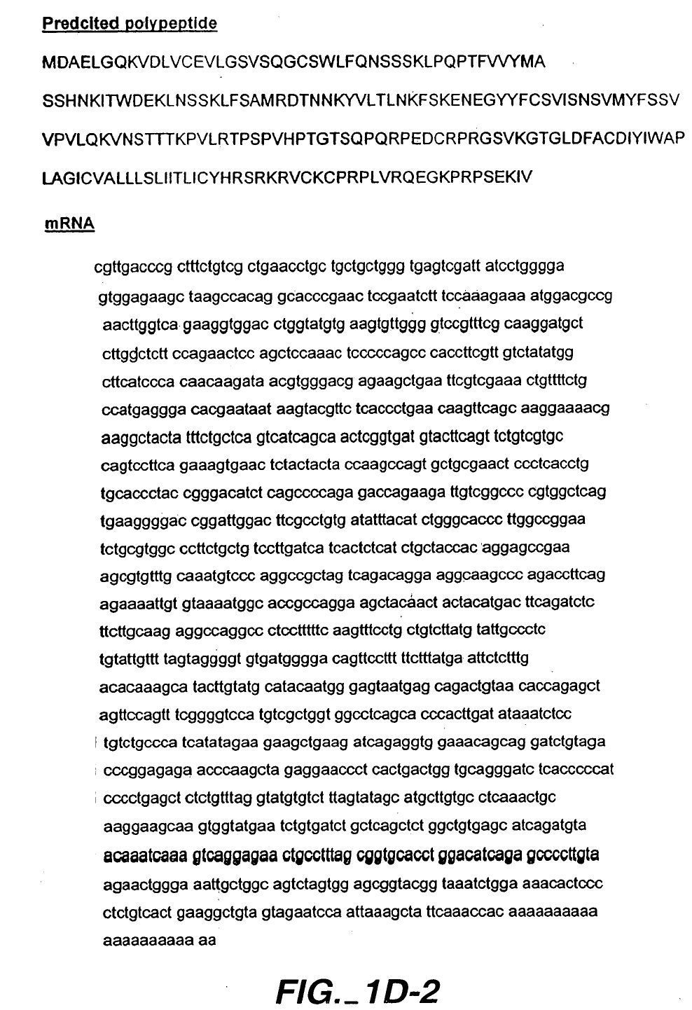

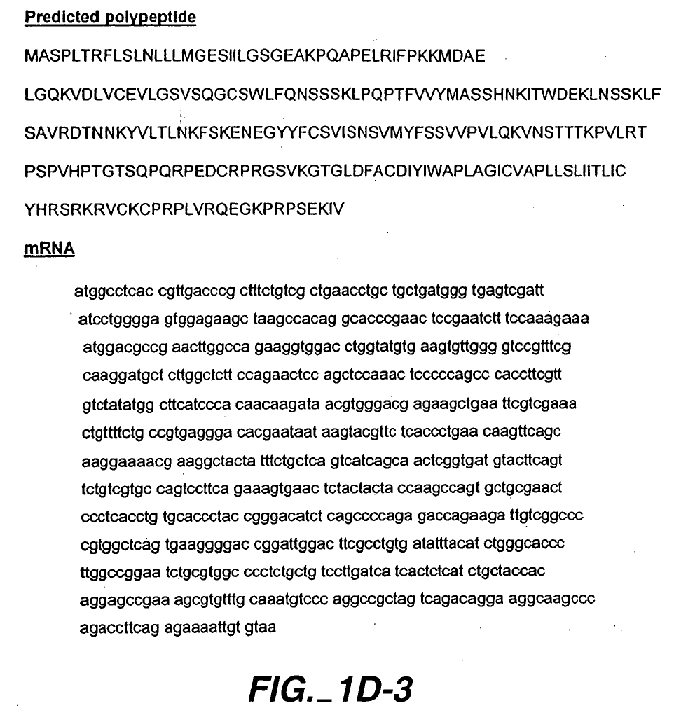

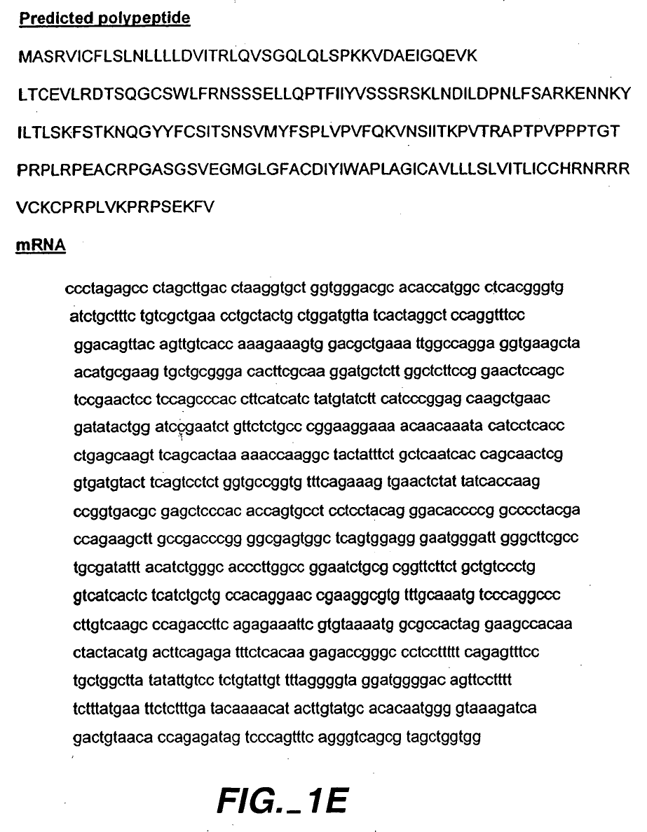

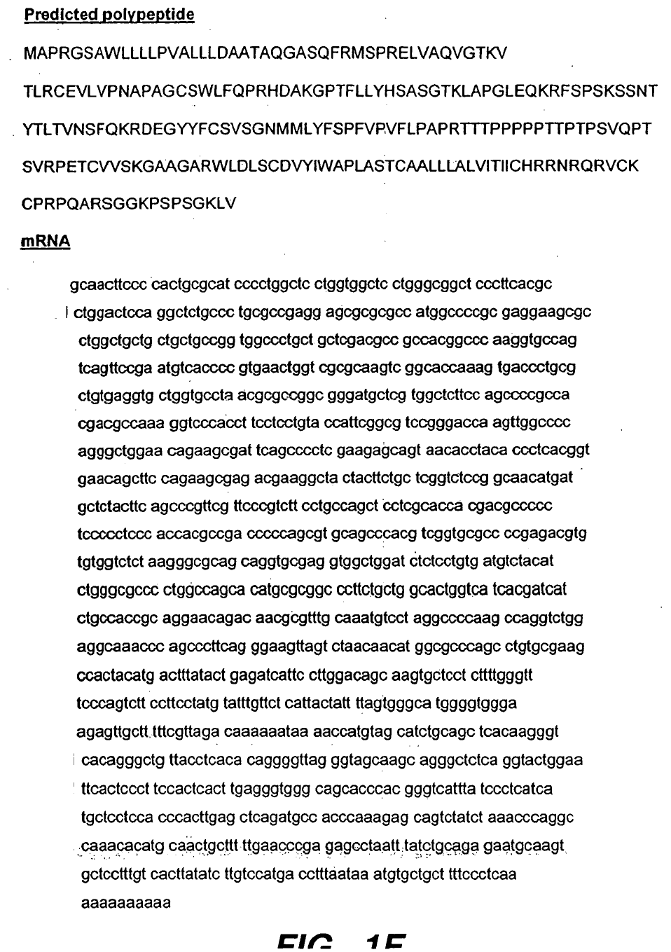

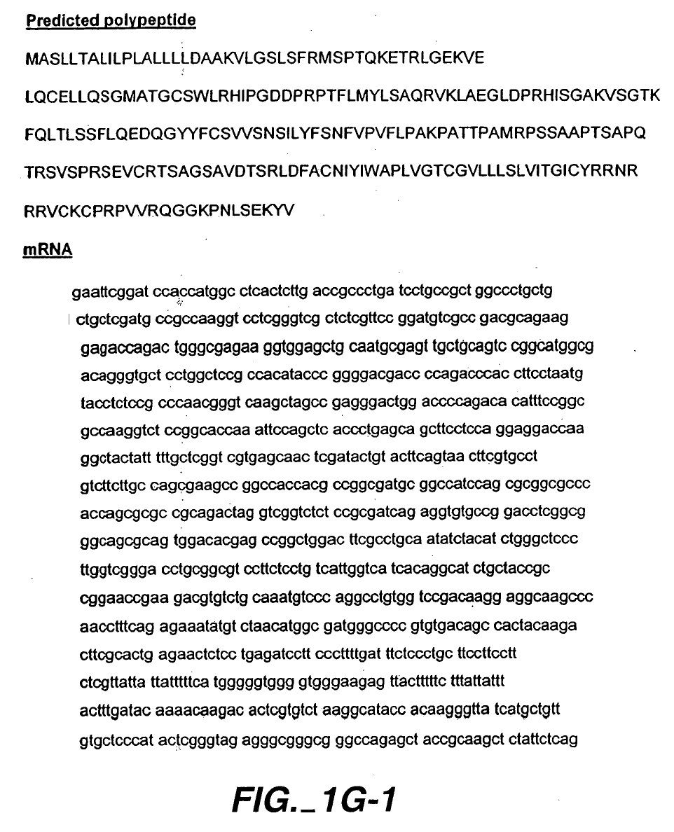

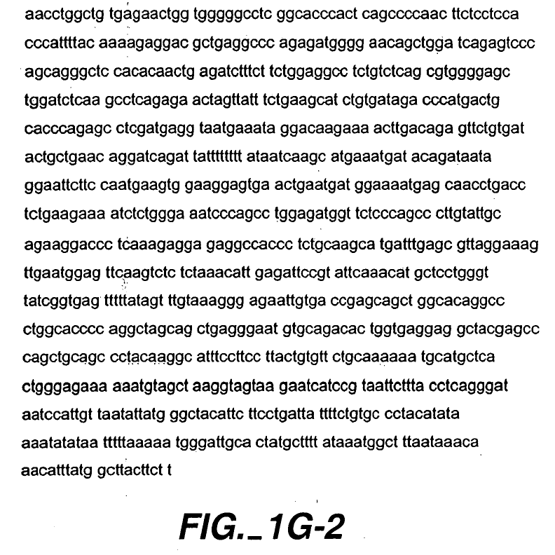

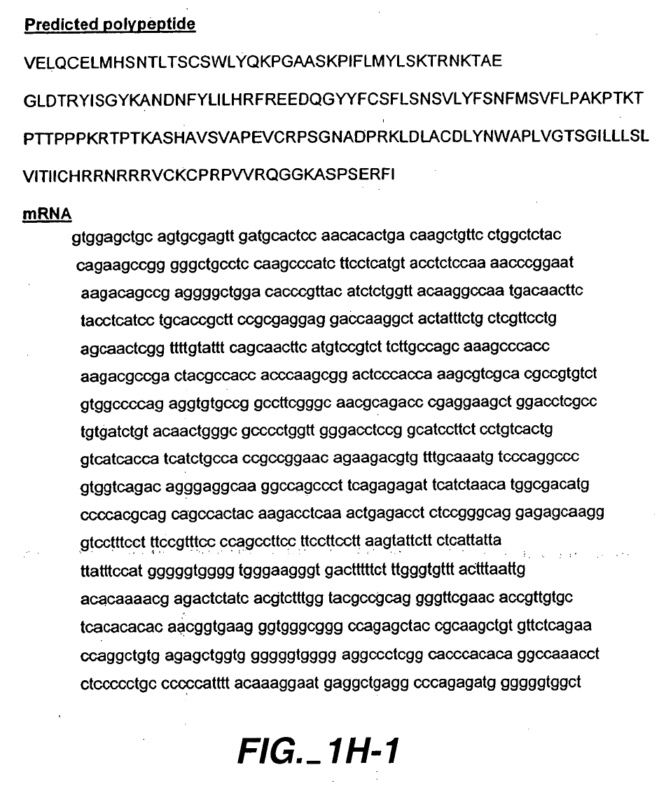

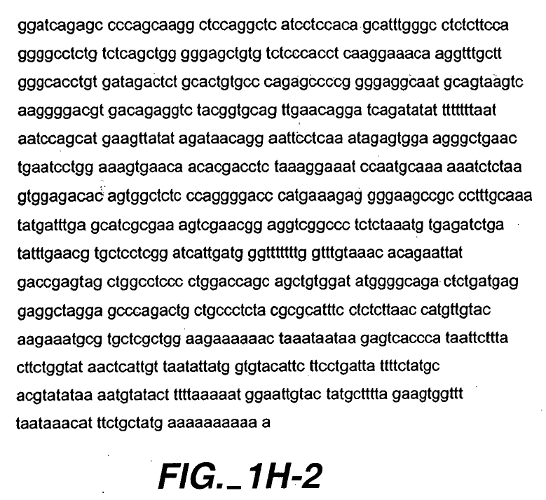

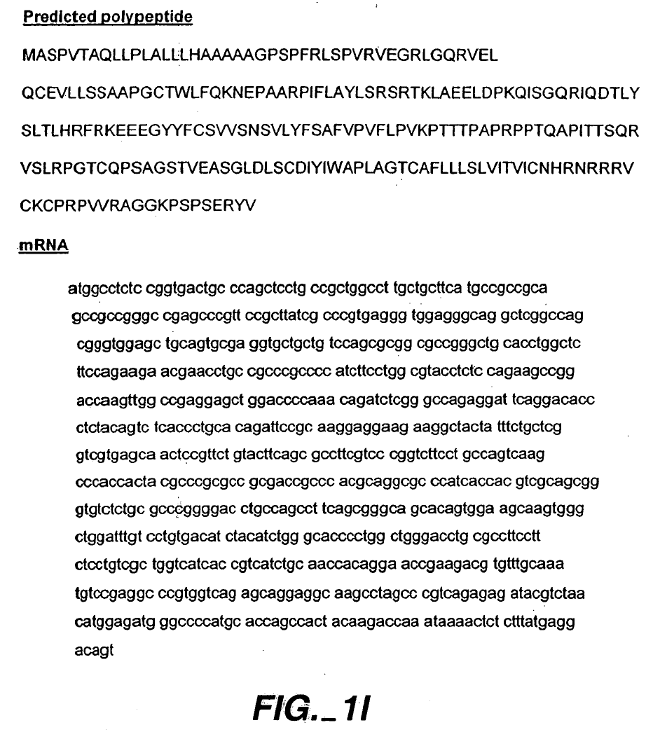

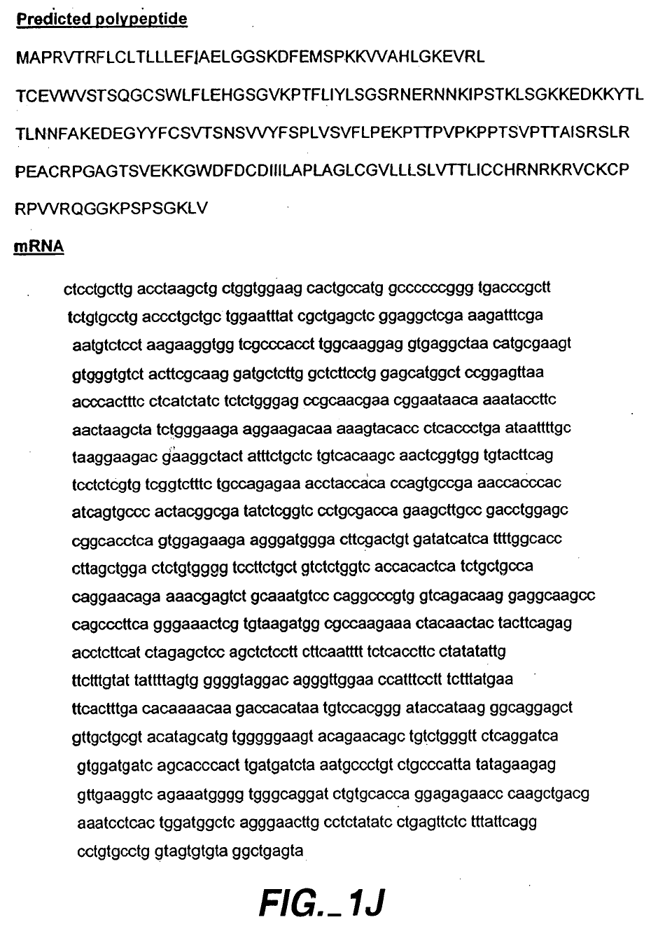

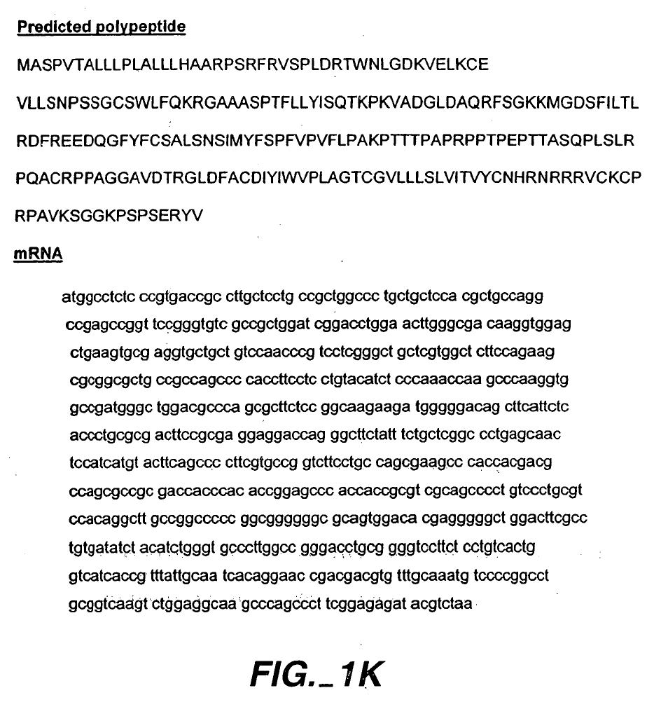

As will be appreciated by those of skill in the art the transmembrane domain of the CD8 α-chain can be exchanged with transmembrane domains of other molecules, if necessary, to modify association of the extracellular domain with the target cell surface. In this embodiment the nucleic acid encoding the extracellular domain of CD8 α-chain is operably linked to a nucleic acid encoding a transmembrane domain. Transmembrane domains of any transmembrane protein can be used in the invention. Alternatively a transmembrane not known to be found in transmembrane proteins. In this embodiment the “synthetic transmembrane domain” contains from around 20 to 25 hydrophobic amino acids followed by at least one and preferably two charged amino acids. In some embodiments the CD8 extracellular domain is linked to the target cell membrane by conventional techniques in the art. Preferred CD8 α-chain sequences are set forth in FIG. 1 and include the full length sequences of either the amino acid sequence or nucleic acid sequence encoding a full length CD8 α-chain from species including human, mouse, rat, orangutan, spider monkey, guinea pig, cow, Hispid cotton rat, domestic pig and cat.

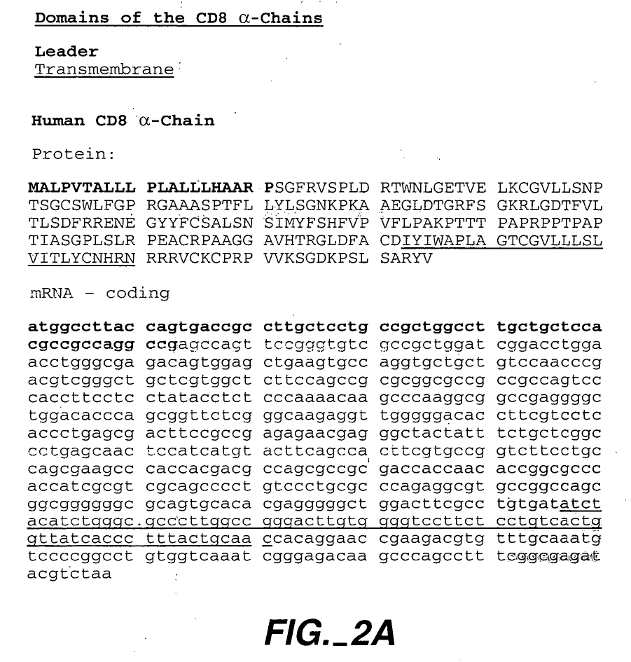

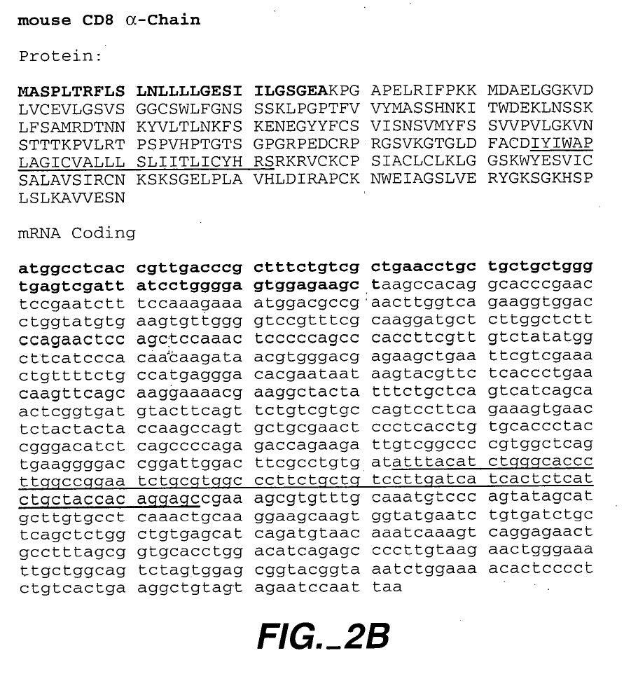

In a preferred embodiment the CD8 α-chain is not a fusion protein, but rather is a truncation protein wherein the intracellular domain is deleted. As depicted in FIG. 2, the human CD8 α-chain gene expresses a protein of 235 amino acids. The protein can be considered to be divided into the following domains (starting at the amino terminal and ending at the carboxy terminal of the polypeptide): a signal peptide (amino acids 1 to 21); immunoglobulin (Ig)-like domain (approximately amino acids 22-136); membrane proximal stalk region (amino acids 137-181); transmembrane domain (amino acids 183-210) and cytoplasmic domain (amino acids 211-235). The nucleotides of the coding sequence that encode these different domains include 1-63 encoding the signal peptide, 64-546 encoding the extracellular domain, about 547-621 encoding the intracellular domain and about 622-708 encoding the intracellular domain. Likewise, the mouse sequences can be divided into domains as follows. The polypeptide can be divided into a signal sequence including amino acids 1-27, an extracellular domain including about amino acids 28 to 194, a transmembrane domain including about amino acids 195-222 and an intracellular domain including about amino acids 223-310. Similarly, the nucleotides of the coding sequence encoding these domain include nucleic acid 1-81 encoding signal peptide, about 82-582 encoding extracellular domain, about 583-666 encoding transmembrane domain and about 667-923 encoding the extracellular domain.

In some embodiments nucleic acid encoding the full length protein is included in the gene delivery vehicle. In other embodiments, nucleic acids encoding the intracellular domain are not included in the polynucleotide in the gene delivery vehicle resulting in a membrane anchored protein lacking the intracellular domain. Corresponding domains also can be identified in other species, including in preferred embodiments the mouse.

One skilled in the art will also appreciate that immunomodulatory molecules having substantial homology to the afore-mentioned polypeptides may find advantageous use in the invention. Accordingly, for example, also encompassed by “CD8 polypeptides” are homologous polypeptides having at least about 80% sequence identity, usually at least about 85% sequence identity, preferably at least about 90% sequence identity, more preferably at least about 95% sequence identity and most preferably at least about 98% sequence identity with the polypeptide encoded by nucleotides shown in FIG. 2.

By “nucleic acid molecules encoding CD8”, and grammatical equivalents thereof is meant the nucleotide sequence of human CD8 as shown in FIG. 2 as well as nucleotide sequences having at least about 80% sequence identity, usually at least about 85% sequence identity, preferably at least about 90% sequence identity, more preferably at least about 95% sequence identity and most preferably at least about 98% sequence identity with nucleotides shown in FIG. 2 and which encode a polypeptide having the sequence shown in FIG. 2, and as set forth in FIG. 1.

As noted previously, a number of different programs can be used to identify whether a protein or nucleic acid has sequence identity or similarity to a known sequence. Sequence identity and/or similarity is determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

Another example of a useful algorithm is the BLAST algorithm, described in Altschul et al., J. Mol. Biol. 215, 403-410, (1990) and Karlin et al., PNAS USA 90:5873-5787 (1993). A particularly useful BLAST program is the WU-BLAST-2 program which was obtained from Altschul et al., Methods in Enzymology, 266: 460480 (1996); http://blast.wustl/edu/blast/README.html]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. Nucleic Acids Res. 25:3389-3402. Gapped BLAST uses BLOSUM-62 substitution scores; threshold T parameter set to 9; the two-hit method to trigger ungapped extensions; charges gap lengths of k a cost of 10+k; Xu set to 16, and Xg set to 40 for database search stage and to 67 for the output stage of the algorithms. Gapped alignments are triggered by a score corresponding to ˜22 bits.

A % amino acid or nucleic acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

The alignment may include the introduction of gaps in the sequences to be aligned. In addition, for sequences which contain either more or fewer amino acids than the amino acid sequence of the polypeptide encoded by nucleotides shown in FIG. 2, it is understood that in one embodiment, the percentage of sequence identity will be determined based on the number of identical amino acids in relation to the total number of amino acids. Thus, for example, sequence identity of sequences shorter than that of the polypeptide encoded by nucleotides in FIG. 2, as discussed below, will be determined using the number of amino acids in the shorter sequence, in one embodiment. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

CD8 having less than 100% sequence identity with the polypeptide encoded by nucleotides in FIG. 2 will generally be produced from native CD8 nucleotide sequences from species other than human and variants of native CD8 nucleotide sequences from human or non-human sources. In this regard, it is noted that many techniques are well known in the art and may be routinely employed to produce nucleotide sequence variants of native CD8 sequences and assaying the polypeptide products of those variants for the presence of at least one activity that is normally associated with a native CD8 polypeptide. In a preferred embodiment the CD8 α-chain is from human but as shown in FIG. 1, CD8 α-chain from rat, mouse, and primates are known and find use in the invention.

Polypeptides having CD8 activity may be shorter or longer than the polypeptide encoded by nucleotides depicted in FIG. 2. Thus, in a preferred embodiment, included within the definition of CD8 polypeptide are portions or fragments of the polypeptide encoded by nucleotides in FIG. 2. In one embodiment herein, fragments of the polypeptide encoded by nucleotides in FIG. 2 are considered CD8 polypeptides if a) they have at least the indicated sequence identity; and b) preferably have a biological activity of naturally occurring CD8, as described above.

In addition, as is more fully outlined below, CD8 α-chain can be made longer than the polypeptide encoded by nucleotides in FIG. 2; for example, by the addition of other fusion sequences, or the elucidation of additional coding and non-coding sequences.

The CD8 polypeptides are preferably recombinant. A “recombinant polypeptide” is a polypeptide made using recombinant techniques, i.e. through the expression of a recombinant nucleic acid as described below. In a preferred embodiment, CD8 of the invention is made through the expression of nucleic acid sequence shown in FIG. 2, or fragment thereof. A recombinant polypeptide is distinguished from naturally occurring protein by at least one or more characteristics. For example, the polypeptide may be isolated or purified away from some or all of the proteins and compounds with which it is normally associated in its wild type host, and thus may be substantially pure. For example, an isolated polypeptide is unaccompanied by at least some of the material with which it is normally associated in its natural state, preferably constituting at least about 0.5%, more preferably at least about 5% by weight of the total protein in a given sample. A substantially pure polypeptide comprises at least about 75% by weight of the total polypeptide, with at least about 80% being preferred, and at least about 90% being particularly preferred. The definition includes the production of a CD8 polypeptide from one organism in a different organism or host cell.

Alternatively, the polypeptide may be made at a significantly higher concentration than is normally seen, through the use of a inducible promoter or high expression promoter, such that the polypeptide is made at increased concentration levels. Alternatively, the polypeptide may be in a form not normally found in nature, as in the addition of amino acid substitutions, insertions and deletions, as discussed below.

In one embodiment, the present invention provides nucleic acid CD8 variants. These variants fall into one or more of three classes: substitutional, insertional or deletional variants. These variants ordinarily are prepared by site specific mutagenesis of nucleotides in nucleotides of FIG. 2, using cassette or PCR mutagenesis or other techniques well known in the art, to produce DNA encoding the variant, including the variant in a gene therapy vector and thereafter expressing the DNA. Amino acid sequence variants are characterized by the predetermined nature of the variation, a feature that sets them apart from naturally occurring allelic or interspecies variation of CD8 amino acid sequence. The variants typically exhibit the same qualitative biological activity as the naturally occurring analogue, although variants can also be selected which have modified characteristics as will be more fully outlined below.

While the site or region for introducing a sequence variation is predetermined, the mutation per se need not be predetermined. For example, in order to optimize the performance of a mutation at a given site, random mutagenesis may be conducted at the target codon or region and the expressed variants screened for the optimal desired activity. Techniques for making substitution mutations at predetermined sites in DNA having a known sequence are well known, for example, M13 primer mutagenesis and PCR mutagenesis. Another example of a technique for making variants is the method of gene shuffling, whereby fragments of similar variants of a nucleotide sequence are allowed to recombine to produce new variant combinations. Examples of such techniques are found in U.S. Pat. Nos. 5,605,703; 5,811,238; 5,873,458; 5,830,696; 5,939,250; 5,763,239; 5,965,408; and 5,945,325, each of which is incorporated by reference herein in its entirety.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger and may include the cytoplasmic domain or fragments thereof.

Substitutions, deletions, insertions or any combination thereof may be used to arrive at a final derivative. Generally these changes are done on a few amino acids to. minimize the alteration of the molecule. However, larger changes may be tolerated in certain circumstances. When small alterations in the characteristics of the CD8 are desired, substitutions are generally made in accordance with the following chart:

CHART 1

| Original | Exemplary |
|---|---|
| Residue | Substitutions |
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart 1. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the CD8 as needed. Alternatively, the variant may be designed such that the biological activity of the protein is altered.

One type of covalent modification of a polypeptide included within the scope of this invention comprises altering the native glycosylation pattern of the polypeptide. “Altering the native glycosylation pattern” is intended for purposes herein to mean deleting one or more carbohydrate moieties found in native sequence CD8 polypeptide, and/or adding one or more glycosylation sites that are not present in the native sequence polypeptide.

Addition of glycosylation sites to polypeptides may be accomplished by altering the amino acid sequence thereof. The alteration may be made, for example, by the addition of, or substitution by, one or more serine or threonine residues to the native sequence polypeptide (for O-linked glycosylation sites). The amino acid sequence may optionally be altered through changes at the DNA level, particularly by mutating the DNA encoding the polypeptide at preselected bases such that codons are generated that will translate into the desired amino acids.

Removal of carbohydrate moieties present on the polypeptide may be accomplished by mutational substitution of codons encoding for amino acid residues that serve as targets for glycosylation.

Once isolated from its natural source, e.g., contained within a plasmid or other vector or excised therefrom as a linear nucleic acid segment, the recombinant nucleic acid can be further-used as a probe to identify and isolate other nucleic acids. It can also be used as a “precursor” nucleic acid to make modified or variant nucleic acids and proteins. It also can be incorporated into a vector or other delivery vehicle for treating target cells as described herein.

Expression Vectors

In the context of the present invention, any suitable expression vector can be used. A “vector” is a vehicle for gene transfer as that term is understood by those of skill in the art. The expression vectors according to the invention include, but are not limited to, plasmids, phages, viruses, liposomes, and the like. An expression vector according to the invention preferably comprises additional sequences and mutations. In particular, an expression vector according to the invention comprises a nucleic acid comprising a transgene encoding an immunomodulatory molecule, particularly a CD8 α-chain, as defined herein. The nucleic acid may comprise a wholly or partially synthetically made coding or other genetic sequence or a genomic or complementary DNA (cDNA) sequence, and can be provided in the form of either DNA or RNA.

A gene encoding for an immunomodulatory molecule can be moved to or from a viral vector or into a baculovirus or a suitable prokaryotic or eukaryotic expression vector for expression of mRNA and production of protein, and for evaluation of other biochemical characteristics.

In terms of the production of vectors according to the invention (including recombinant adenoviral vectors and transfer vectors), such vectors can be constructed using standard molecular and genetic techniques, such as those known to those skilled in the art. Vectors comprising virions or viral particles (e.g., recombinant adenoviral vectors) can be produced using viral vectors in the appropriate cell lines. Similarly, particles comprising one or more chimeric coat proteins can be produced in standard cell lines, e.g., those currently used for adenoviral vectors. These resultant particles then can be targeted to specific cells, if desired.

Any appropriate expression vector (e.g., as described in Pouwels et al., Cloning Vectors: A Laboratory Manual (Elsevior, N.Y.: 1985)) and corresponding suitable host cell can be employed for production of a recombinant peptide or protein in a host cell. Expression hosts include, but are not limited to, bacterial species within the genera Escherichia, Bacillus, Pseudomonas, Salmonella, mammalian or insect host cell systems, including baculoviral systems (e.g., as described by Luckow et al., Bio/Technology, 6, 47 (1988)), and established cell lines, such as COS-7, C127, 3T3, CHO, HeLa, BHK, and the like. An especially preferred expression system for preparing chimeric proteins (peptides) according to the invention is the baculoviral expression system wherein Trichoplusia ni, Tn 5B1-4 insect cells, or other appropriate insect cells, are used to produce high levels of recombinant proteins. The ordinary skilled artisan is, of course, aware that the choice of expression host has ramifications for the type of peptide produced. For instance, the glycosylation of peptides produced in yeast or mammalian cells (e.g., COS-7 cells) will differ from that of peptides produced in bacterial cells, such as Escherichia coli.

In a preferred embodiment, the proteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for a protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

The protein may also be made as a fusion protein, using techniques well known in the art. Thus, for example, the protein may be made as a fusion protein to increase expression, or for other reasons. For example, when the protein is a peptide, the nucleic acid encoding the peptide may be linked to other nucleic acid for expression purposes.

To test for CD8, the protein is purified or isolated after expression. Proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the CD8 protein may be purified using a standard anti-CD8 antibody column. Ultrafiltration and diafiltration techniques, in conjunction with protein concentration, are also useful. For general guidance in suitable purification techniques, see Scopes, R., Protein Purification, Springer-Verlag, N.Y. (1982). The degree of purification necessary will vary depending on the use of the CD8 protein. In some instances no purification will be necessary. in some instances CD8 expression is detected on the cell surface, for example by antibody binding and detection via fluorescence or by Fluorescence Activated Cell Sorting (FACS).

Nucleic acid molecules encoding CD8 as well as any nucleic acid molecule derived from either the coding or non-coding strand of a CD8 nucleic acid molecule may be contacted with cells of an target in a variety of ways that are known and routinely employed in the art, wherein the contacting may be ex vivo or in vivo.

Viral attachment, entry and gene expression can be evaluated initially by using the adenoviral vector containing the insert of interest to generate a recombinant virus expressing the desired protein or RNA and a marker gene, such as β-galactosidase. β-galactosidase expression in cells infected with adenovirus containing the β-galactosidase gene (Ad-LacZ) can be detected as early as two hours after adding Ad-Gluc to cells. This procedure provides a quick and efficient analysis of cell entry of the recombinant virus and gene expression, and is implemented readily by an artisan of ordinary skill using conventional techniques.

Using the nucleic acids of the present invention which encode a protein, a variety of expression vectors can be made. The expression vectors may be either self-replicating extrachromosomal vectors or vectors which integrate into a host genome. Generally, these expression vectors include transcriptional and translational regulatory nucleic acid operably linked to the nucleic acid encoding the protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. As another example, operably linked refers to DNA sequences linked so as to be contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the CD8; for example, human transcriptional and translational regulatory nucleic acid sequences are preferably used to express the CD8 in human cells. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In a further embodiment, the expression vector may contain a selectable marker gene to allow the selection of transformed host cells. Selection genes are well known in the art and will vary with the host cell used.

Preferably, the vector is a viral vector, such as an adenoviral vector, an adeno-associated viral vector, a herpes vector or a retroviral vector, among others. Most preferably, the viral vector is an adenoviral vector. An adenoviral vector can be derived from any adenovirus. An “adenovirus” is any virus of the family Adenoviridae, and desirably is of the genus Mastadenovirus (e.g., mammalian adenoviruses) or Aviadenovirus (e.g., avian adenoviruses). The adenovirus is of any serotype. Adenoviral stocks that can be employed as a source of adenovirus can be amplified from the adenoviral serotypes 1 through 47, which are currently available from the American Type Culture Collection (ATCC, Rockville, Md.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype. Preferably, however, an adenovirus is of serotypes 2, 5 or 9. Desirably, an adenovirus comprises coat proteins (e.g., penton base, hexon, and/or fiber) of the same serotype. However, also preferably, one or more coat proteins can be chimeric, in the sense, for example, that all or a part of a given coat protein can be from another serotype.

Although the viral vector, which is preferably an adenoviral vector, can be replication-competent, preferably, the viral vector is replication-deficient or conditionally replication-deficient. For example, the viral vector which is preferably an adenoviral vector, comprises a genome with at least one modification that renders the virus replication-deficient. The modification to the viral genome includes, but is not limited to, deletion of a DNA segment, addition of a DNA segment, rearrangement of a DNA segment, replacement of a DNA segment, or introduction of a DNA lesion. A DNA segment can be as small as one nucleotide or as large as 36 kilobase pairs, i.e., the approximate size of the adenoviral genome, or 38 kilobase pairs, which is the maximum amount that can be packaged into an adenoviral virion.

Preferred modifications to the viral, in particular adenoviral, genome include, in addition to a modification that renders the virus replication-deficient, the insertion of a transgene encoding for an immunomodulatory molecule as defined herein and, additionally and preferably, at least one transgene encoding for a therapeutic molecule of interest. A virus, such as an adenovirus, also preferably can be a cointegrate, i.e., a ligation of viral, such as adenoviral, genomic sequences with other sequences, such as those of a plasmid, phage or other virus.

In terms of an adenoviral vector (particularly a replication-deficient adenoviral vector), such a vector can comprise either complete capsids (i.e., including a viral genome, such as an adenoviral genome) or empty capsids (i.e., in which a viral genome is lacking, or is degraded, e.g., by physical or chemical means). Preferably, the viral vector comprises complete capsids, i.e., as a means of carrying the transgene encoding for the immunomodulatory molecule and, optionally and preferably, at least one transgene encoding an inhibiting means. Alternatively, preferably, the transgenes may be carried into a cell on the outside of the adenoviral capsid.

To the extent that it is preferable or desirable to target a virus, such as an adenovirus, to a particular cell, the virus can be employed essentially as an endosomolytic agent in the transfer into a cell of plasmid DNA, which contains a marker gene and is complexed and condensed with polylysine covalently linked to a cell-binding ligand, such as transferrin (Cotten et al., PNAS (USA), 89, 6094-6098 (1992); and Curiel et al., PNAS (USA), 88, 8850-8854 (1991)). It has been demonstrated that coupling of the transferrin-polylysine/DNA complex and adenovirus (e.g., by means of an adenovirus-directed antibody, with transglutaminase, or via a biotin/streptavidin bridge) substantially enhances gene transfer (Wagner et al., PNAS (USA), 89, 6099-6103 (1992)).

Alternatively, one or more viral coat proteins, such as the adenoviral fiber, can be modified, for example, either by incorporation of sequences for a ligand to a cell-surface receptor or sequences that allow binding to a bispecific antibody (i.e., a molecule with one end having specificity for the fiber, and the other end having specificity for a cell-surface receptor) (PCT international patent application no. WO 95/26412 (the '412 application) and Watkins et al., “Targeting Adenovirus-Mediated Gene Delivery with Recombinant Antibodies,” Abst. No. 336). In both cases, the typical fiber/cell-surface receptor interactions are abrogated, and the virus, such as an adenovirus, is redirected to a new cell-surface receptor by means of its fiber.

Alternatively, a targeting element, which is capable of binding specifically to a selected cell type, can be coupled to a first molecule of a high affinity binding pair and administered to a host cell (PCT international patent application no. WO 95/31566). Then, a gene delivery vehicle coupled to a second molecule of the high affinity binding pair can be administered to the host cell, wherein the second molecule is capable of specifically binding to the first molecule, such that the gene delivery vehicle is targeted to the selected cell type.

Along the same lines, since methods (e.g., electroporation, transformation, conjugation of triparental mating, (co-)transfection, (co-) infection, membrane fusion, use of microprojectiles, incubation with calcium phospate-DNA precipitate, direct microinjection; etc.) are available for transferring viruses, plasmids, and phages in the form of their nucleic acid sequences (i.e., RNA or DNA), a vector similarly can comprise RNA or DNA, in the absence of any associated protein, such as capsid protein, and in the absence of any envelope lipid.

Similarly, since liposomes effect cell entry by fusing with cell membranes a vector can comprise liposomes, with constitutive nucleic acids encoding the coat protein. Such liposomes are commercially available, for instance, from Life Technologies, Bethesda, Md., and can be used according to the recommendation of the manufacturer. Moreover, a liposome can be used to effect gene delivery and liposomes having increased tranfer capacity and/or reduced toxicity in vivo can be used. The soluble chimeric coat protein (as produced using methods described herein) can be added to the liposomes either after the liposomes are prepared according to the manufacturer's instructions, or during the preparation of the liposomes.

The vectors according to the invention are not limited to those that can be employed in the method of the invention, but also include intermediary-type vectors (e.g., “transfer vectors”) that can be employed in the construction of gene transfer vectors.

One of the preferred methods for in vivo delivery of one or more nucleic acid sequences involves the use of an adenovirus expression vector. “Adenovirus expression vector” is meant to include those constructs containing adenovirus sequences sufficient to (a) support packaging of the construct and (b) to express a polynucleotide that has been cloned therein in a sense or antisense orientation. Of course, in the context of an antisense construct, expression does not require that the gene product be synthesized.

The expression vector comprises a genetically engineered form of an adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the adenovirus vectors, which are replication deficient, depend on a unique helper cell line. In nature, adenovirus can package approximately 105% of the wild-type genome (Ghosh-Choudhury et al., 1987), providing capacity for about 2 extra kB of DNA. Combined with the approximately 5.5 kB of DNA that is replaceable in the E1 and E3 regions, the maximum capacity of the current adenovirus vector is under 7.5 kB, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the currently preferred helper cell line is 293.

Recently, Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

In a preferred embodiment the adenovirus is a “gutless” adenovirus as is known in the art. The “gutless” adenovirus vector is a recently developed system for adenoviral gene delivery. The replication of the adenovirus requires a helper virus and a special human 293 cell line expressing both E1a and Cre, a condition that does not exist in natural environment. In the most efficient system to date, an E1-deleted helper virus is used with a packaging signal that is flanked by bacteriophage P1 loxP sites (“floxed”). Infection of the helper cells that express Cre recombinase with the gutless virus together with the helper virus with a floxed packaging signal should only yield gutless rAV, as the packaging signal is deleted from the DNA of the helper virus. However, if 293-based helper cells are used, the helper virus DNA can recombine with the Ad5 DNA that is integrated in the helper cell DNA. As a result, a wild-type packaging signal, as well as the E1 region, is regained. Thus, also production of gutless rAV on 293- (or 911-) based helper cells can result in the generation of RCA, if an E1-deleted helper virus is used.

The vector is deprived of all viral genes. Thus the vector is non-immunogenic and may be used repeatedly, if necessary. The “gutless” adenovirus vector also contains 36 kb space for accommodating transgenes, thus allowing co-delivery of a large number of genes into cells. Specific sequence motifs such as the RGD motif may be inserted into the H-1 loop of an adenovirus vector to enhance its infectivity. An adenovirus recombinant is constructed by cloning specific transgenes or fragments of transgenes into any of the adenovirus vectors such as those described herein and known in the art. The adenovirus recombinant can be used to transduce epidermal cells of a vertebrate in a non-invasive mode for use as an immunizing agent.

Use of the “gutless” adenoviruses is particularly advantageous for insertion of large inserts of heterologous DNA (for a review, see Yeh. and Perricaudet, FASEB J. 11:615 (1997)), which is incorporated herein by reference. In addition, gutless adenoviral vectors and methods of making and using them are described in more detail in U.S. Pat. Nos. 6,156,497 and 6,228,646, both of which are expressly incorporated herein by reference.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain a conditional replication-defective adenovirus vector for use in the present invention, since Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transgene encoding the immunomodulatory molecule and/or additional therapeutic protein of interest at the position from which the E1-coding sequences have been removed. However, the position of insertion of the expression construct within the adenovirus sequences is not critical to the invention. The transgene(s) of interest may also be inserted in lieu of the deleted E3 region in E3 replacement vectors as described by Karlsson et al. (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 109 -1011 plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

Accordingly, in a preferred embodiment, the expression vectors used herein are adenoviral vectors. Suitable adenoviral vectors include modifications of human adenoviruses such as Ad2 or Ad5, wherein genetic elements necessary for the virus to replicate in vivo have been removed; e.g. the E1 region, and an expression cassette coding for the exogenous gene of interest inserted into the adenoviral genome.

In addition, as described above, a preferred expression vector system is a retroviral vector system such as is generally described in PCT/US97/01019 and PCT/US97/01048, both of which are hereby expressly incorporated by reference.

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding one or more oligonucleotide or polynucleotide sequences of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class 11 antigens, they demonstrated the infection of a variety of human cells that bore those surface antigens with an ecotropic virus in vitro (Roux et al., 1989). Suitable retroviral vectors include LNL6, LXSN, and LNCX (see Byun et al., Gene Ther. 3(9):780-8 (1996 for review).

AAV (Ridgeway, 1988; Hermonat and Muzycska, 1984) is a parvovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Five serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter (Muzyczka and McLaughlin, 1988).

The AAV DNA is approximately 4.7.kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three viral promoters have been identified and named p5, p19, and p40, according to their map position. Transcription from p5 and p19 results in production of rep proteins, and transcription from p40 produces the capsid proteins (Hermonat and Muzyczka, 1984).

AAV is also a good choice of delivery vehicles due to its safety. There is a relatively complicated rescue mechanism: not only wild type adenovirus but also AAV genes are required to mobilize rAAV. Likewise, AAV is not pathogenic and not associated with any disease. The removal of viral coding sequences minimizes immune reactions to viral gene expression, and therefore, rAAV does not evoke an inflammatory response. Other disclosure related to AAV is set forth in U.S. Pat. No. 6,531,456, which is expressly incorporated herein by reference.

Other viral vectors may be employed as expression vectors in the present invention for the delivery of immunomodulatory molecules to a host cell. Vectors derived from viruses such as vaccinia virus (Ridgeway, 1988; Coupar et al., 1988), lentiviruses, polio viruses and herpes viruses may be employed. They offer several attractive features for various mammalian cells (Friedmann, 1989; Ridgeway, 1988; Coupar et al., 1988; Horwich et al., 1990).

Delivery of Expression Vectors

In order to effect expression of the immunomodulatory molecule (e.g. CD8 α-chain) and/or additional therapeutic protein the expression vectors must be delivered into a cell. This delivery may be accomplished in vitro, as in laboratory procedures for transforming cells lines, or in vivo or ex vivo, as in the treatment of certain disease states. As described above, one preferred mechanism for delivery is via infection where the nucleic acid is encapsulated in a recombinant viral particle.

Once the expression vector has been delivered into the cell the nucleic acid encoding the desired oligonucleotide or polynucleotide sequences may be positioned and expressed at different sites. In certain embodiments, the nucleic acid encoding the construct may be stably integrated into the genome of the cell. This integration may be in the specific location and orientation via homologous recombination (gene replacement) or it may be integrated in a random, non-specific location (gene augmentation). In further and preferred embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression vector employed.

In certain embodiments of the invention, the expression vector may simply consist of naked recombinant DNA or plasmids. Transfer of the vector may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Reshef (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have generally consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e. ex vivo treatment. Again, DNA encoding a particular gene may be delivered via this method and still be incorporated by the present invention.

In one embodiment of the present invention, the nucleic acid molecule is introduced into target cells, by liposome-mediated nucleic acid transfer. In this regard, many liposome-based reagents are well known in the art, are commercially available and may be routinely employed for introducing a nucleic acid molecule into cells of the target. Certain embodiments of the present invention will employ cationic lipid transfer vehicles such as Lipofectamine or Lipofectin (Life Technologies), dioleoylphosphatidylethanolamine (DOPE) together with a cationic cholesterol derivative (DC cholesterol), N[1-(2,3-dioleyloxy)propyl]-N,N,N-trimethylammonium chloride (DOTMA) (Sioud et al., J. Mol. Biol. 242:831-835 (1991)), DOSPA:DOPE, DOTAP, DMRIE:cholesterol, DDAB:DOPE, and the like. Production of liposome-encapsulated nucleic acid is well known in the art and typically involves the combination of lipid and nucleic acid in a ratio of about 1:1.

Uses of the Present Invention

As detailed above, the methods and compositions described and enabled herein find general utility in inhibiting an alloimmune response to donor and/or recipient antigens for use, e.g., in transplantation and treatment of GVHD. According to the present invention, allograft survival can be extended without the need for chronic general immunosuppressive agents by conditioning the allograft cells to express a CD8 polypeptide, and more preferably, the CD8 α-chain. Targeted expression of the CD8 polypeptide as described herein results in effective and specific inhibition of the recipient immune response directed to donor antigens in the allograft. Conversely, conditioning of recipient cells at risk of GVHD to express a CD8 polypeptide results in effective and specific inhibition of a GVHD immune response directed to such recipient cells by donor T cells from the allograft, and thus GVHD may be prevented and/or treated thereby.

Without being bound by theory, it is thought that expression of CD8 on target cells confers on the target cells the ability to induce the “veto effect” on a host immune system. That is, as described above, when cells expressing CD8 are contacted with host T cells, the T cells are downregulated or killed. Accordingly, by “veto” or “veto effect” is meant the ability of a target cell to downregulate the immune response against the target cell. It is thought that CD8 is necessary for induction or transfer of the veto effect, and in particular, the CD8 α-chain. By “transfer of the veto effect” is meant that the veto effect is transferred to a cell that normally would not induce the veto effect. That is, the ability to reduce or downregulate the immune response to a target cell is conferred upon the target cell by induced or increased expression of CD8. As reported for the first time herein, it has now been surprisingly discovered that the presence of CD8 α-chain on target cells can “veto” the activity of CD4+ T-cells as well as CD8+ cells, and thus both the cellular and humoral components of the immune response may be inhibited thereby.

Accordingly, the invention finds use in reducing the immune response to target cells by inducing the veto effect. This results in the down regulation and deletion of T cells that would otherwise recognize the target cell. When the target cell is a cell expressing an autoimmune antigen, inducing the veto effect protects against a host autoimmune response. When the target cell is a stem cell for use, e.g., in a transplant scenario to repopulate a particular cell type, inducing the veto effect against the stem cell protects the population of stem cells. When the target cell is an allograft cell, e.g., transplant tissue, inducing the veto effect protects against rejection by reducing or down-regulating the immune response against non self-antigens or alloantigens expressed by or present in the allograft. Likewise, it is also contemplated that the methods and compositions provided herein will find advantageous use in the field of xenotransplantation, by inhibiting the immune response raised against xenoantigens in organs, tissues and cells transplanted from non-human mammals such as, e.g., porcine, equine, non-human primates and the like.

Generally, when expression of CD8 is used in transplant scenario, the graft life will be extended for a significant amount of time beyond what could normally be anticipated in the absence of the subject nucleic acids, more usually at least five days, more preferably at least about 30 days, and even more preferably about 3 months and most preferably about 6 months to one year. The actual amount of time transplant life is extended will vary with the various conditions of the procedure, particularly depending on the organ type to be transplanted. Also, treatment of the target cell with the delivery vehicle containing the CD8 nucleic acid can be repeated if CD8 expression declines such that the target cell is recognized by the host immune response. This also can be useful in areas where xenogeneic grafts have been used awaiting an allogeneic graft, to allow for reduced amounts of general immunosuppressive agents or avoid using such immunosuppressants altogether.

An expression vector of the present invention therefore has utility in vitro. Such a vector can be used as a research tool in the study of viral clearance and persistence and in a method of assessing the efficacy of means of circumventing an immune response. Similarly, an expression vector, preferably a recombinant expression vector, specifically a viral or adenoviral vector, which comprises a transgene and at least one gene encoding for an immunomodulatory molecule, can be employed in vivo.

In vivo delivery includes, but is not limited to direct injection into the organ, via catheter, or by other means of perfusion. The nucleic acid may be administered intravascularly at a proximal location to the transplant organ or administered systemically. One of ordinary skill in the art will recognized the advantages and disadvantages of each mode of delivery. For instance, direct injection may produce the greatest titer of nucleic acid in the organ, but distribution of the nucleic acid will likely be uneven throughout the organ. Introduction of the nucleic acid proximal to the transplant organ will generally result in greater contact with the cells of the organ, but systemic administration is generally much simpler. Administration may also be to the donor prior to removal of the organ. The nucleic acids may be introduced in a single administration, or several administrations, beginning before removal of the organ from the donor as well as after transplantation. The skilled artisan will be able to determine a satisfactory means of delivery and delivery regimen without undue experimentation.

Nucleic acids may be contacted with cells of the transplant organ ex vivo using methods well known to the skilled artisan. As described herein, conventional organ preservation solutions can be considerably improved through the addition of the expression vectors detailed herein. The temperature at which the organ may be maintained will be conventional, typically in the range of about 1° to 8° C. The residence time of the organ in the medium will generally be in the range of about 10 minutes to 48 hours, more usually about 10 minutes to 2 hours. The nucleic acids may be contacted with cells of the organ in vivo as well as ex vivo.

In a preferred embodiment, the nucleic acid is contacted with cells of an organ transplant by direct injection into the transplanted organ. In this regard, it is well known in the art that living cells are capable of internalizing and incorporating exogenous nucleic acid molecule with which the cells come in contact. That nucleic acid may then be expressed by the cell that has incorporated it into its nucleus. In a preferred embodiment, the nucleic acid is contacted with cells of a transplant organ by intravascular injection proximate to the transplant organ. In an alternate preferred embodiment, the nucleic acid is contacted with cells of a transplant organ by systemic administration.

The subject nucleic acids may be used with a wide variety of hosts, particularly primates, more particularly humans, or with domestic animals. The subject nucleic acids may be used in conjunction with the transplantation of a wide variety of organs, including, but not limited to, kidney, heart, liver, spleen, bone marrow, pancreas, lung, and islet of Langerhans. The subject nucleic acids may be used for allogeneic, as well as xenogeneic, grafts.

Expression vectors, such as recombinant adenoviral vectors, of the present invention can also be used to treat any one of a number of diseases by delivering to cells corrective DNA, e.g., DNA encoding a function that is either absent or impaired. Diseases that are candidates for such treatment include, for example, cancer, e.g., melanoma or glioma, cystic fibrosis, genetic disorders, and pathogenic infections, including HIV infection. See, e.g., co-pending U.S. patent application Ser. No. XX, incorporated by reference herein. In further preferred embodiments, vectors capable of expressing an immunomodulatory molecule (e.g. CD8) with or without an additional therapeutic molecules can be advantageously used to inhibit host immune responses against transplanted tissue, or prevent autoimmune responses to self antigens. Other applications of the method and compositions of the present invention will be apparent to those skilled in the art.

Formulations and Dosing of Expression Vectors

One skilled in the art will appreciate that many suitable methods of administering an expression vector (particularly an adenoviral vector) to an animal (see, for example, Rosenfeld et al., Science, 252, 431-434 (1991); Jaffe et al., Clin. Res., 39(2), 302A (1991); Rosenfeld et al., Clin. Res., 39(2), 31 1A (1991); Berkner, BioTechniques, 6, 616-629 (1988)) are available, and, although more than one route can be used for administration, a particular route can provide a more immediate and more effective reaction than another route. Pharmaceutically acceptable excipients for use in administering the expression vector and/or means of inhibiting an immune response also are well-known to those who are skilled in the art, and are readily available. The choice of excipient will be determined in part by the particular method used to administer the expression vector and for means of inhibiting an immune response. Accordingly, the present invention provides a composition comprising an expression vector encoding an immunomodulatory molecule (e.g. CD8 α-chain), alone or in further combination with a transgene, in a suitable carrier, and there are a wide variety of suitable formulations for use in the context of the present invention. In particular, the present invention provides a composition comprising an expression vector comprising a gene encoding an alpha chain of CD8 (or a functional fragment thereof and a carrier therefore. In alternative embodiments, the expression vector further encodes a therapeutic molecule or protein of interest such as, e.g., an anti-inflammatory molecule. Such compositions can further comprise other active agents, such as therapeutic or prophylactic agents and/or immunosuppressive agents as are known in the art. The following methods and excipients are merely exemplary and are in no way limiting.

Formulations suitable for oral administration can consist of (a) liquid solutions, such as an effective amount of the compound dissolved in diluents, such as water, saline, or orange juice; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, emulsions, gels, and the like containing, in addition to the active ingredient, such excipients as are known in the art.

Aerosol formulations can be made for administration via inhalation. These aerosol formulations can be placed into pressurized acceptable propellants, such as dichlorodifluoromethane, propane, nitrogen, and the like. They also can be formulated as pharmaceuticals for non-pressurized preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Additionally, suppositories can be made with the use of a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

The dose administered to an animal, particularly a human, in the context of the present invention will vary with the therapeutic transgene of interest, source of vector and/or the nature of the immunomodulatory molecule, the composition employed, the method of administration, and the particular site and organism being treated. However, preferably, a dose corresponding to an effective amount of a vector (e.g., an adenoviral vector according to the invention) is employed. An “effective amount” is one that is sufficient to produce the desired effect in a host, which can be monitored using several end-points known to those skilled in the art. For instance, one desired effect is nucleic acid transfer to a host cell. Such transfer can be monitored by a variety of means, including, but not limited to, a therapeutic effect (e.g., alleviation of some symptom associated with the disease, condition, disorder or syndrome being treated), or by evidence of the transferred gene or coding sequence or its expression within the host (e.g., using the polymerase chain reaction, Northern or Southern hybridizations, or transcription assays to detect the nucleic acid in host cells, or using immunoblot analysis, antibody-mediated detection, or particularized assays to detect protein or polypeptide encoded by the transferred nucleic acid, or impacted in level or function due to such transfer). These methods described are by no means all-inclusive, and further methods to suit the specific application will be apparent to the ordinary skilled artisan. In this regard, it should be noted that the response of a host to the introduction of a vector, such as a viral vector, in particular an adenoviral vector, as well as a vector encoding a means of inhibiting an immune response, can vary depending on the dose of virus administered, the site of delivery, and the genetic makeup of the vector as well as the transgene and the means of inhibiting an immune response.

Generally, to ensure effective transfer of the vectors of the present invention, it is preferable that about 1 to about 5,000 copies of the vector according to the invention be employed per cell to be contacted, based on an approximate number of cells to be contacted in view of the given route of administration, and it is even more preferable that about 3 to about 300 pfu enter each cell. However, this is merely a general guideline, which by no means precludes use of a higher or lower amount, as might be warranted in a particular application, either in vitro or in vivo. Similarly, the amount of a means of inhibiting an immune response, if in the form of a composition comprising a protein, should be sufficient to inhibit an immune response to the recombinant vector comprising the transgene. For example, the actual dose and schedule can vary depending on whether the composition is administered in combination with other pharmaceutical compositions, or depending on interindividual differences in pharmacokinetics, drug disposition, and metabolism. Similarly, amounts can vary in in vitro applications, depending on the particular cell type targeted or the means by which the vector is transferred. One skilled in the art easily can make any necessary adjustments in accordance with the necessities of the particular situation.

Although the present invention has been described with reference to preferred embodiments, persons skilled in the art will recognize that changes may be made in form and detail without departing from the spirit and scope of the invention. Each of the patents, publications and other references identified herein is expressly incorporated by reference herein in its entirety.

### EXAMPLE 1

### The Veto Effect—STUDIES WITH VECTORS

a. The Use of Plasmid Expression Vectors to Engineer Fibroblasts as Veto Cells

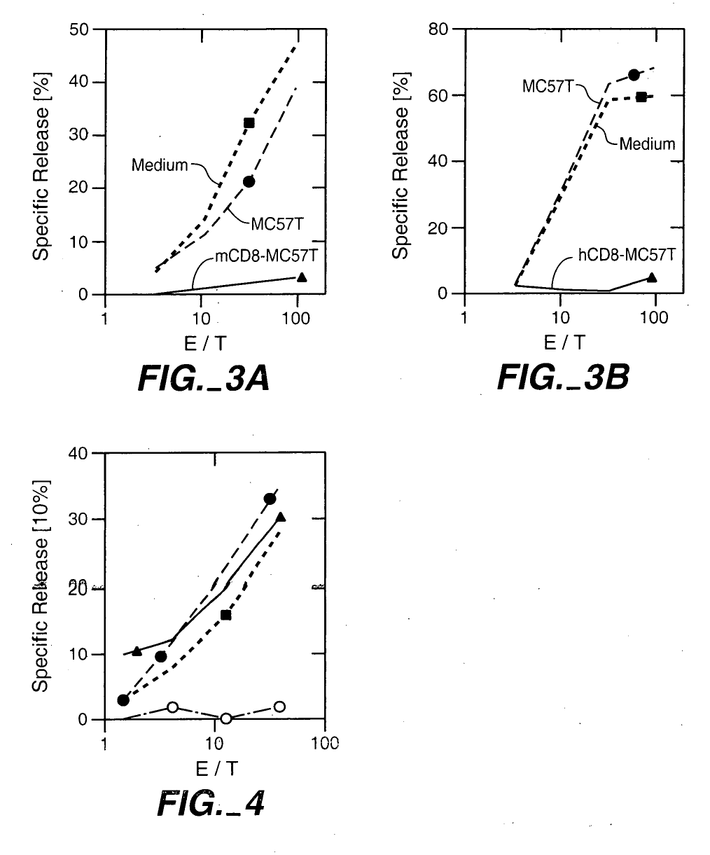

Fibroblasts were engineered to express either human or mouse CD8 α-chain on their surface. Fibroblasts were transfected with the pCMVhCD8α plasmid or pCMVmCD8α plasmid in which expression of the CD8 α-chain is driven by the CMV immediate early promotor/enhancer (Invitrogen). When the CD8 α-chain transfected fibroblasts (H-2^{b}) were added to mixed lymphocyte cultures (Balb/c; H-2^{d} anti-C57BL/6; H-2^{b}), only the CD8 α-chain expressing line suppressed CTL responses. As depicted in FIGS. 3A and B, the addition of MC57T fibroblasts expressing either the mouse or human CD8 α-chain completely suppressed the induction of CTLs. In contrast, the addition of non-transfected fibroblasts did not affect T-lymphocyte activation. In addition to establishing the inhibitory function of a CD8 α-chain, these experiments also demonstrated that mouse T-lymphocytes could be veto-ed with the human CD8 α-chain. Therefore, the mouse model will be useful in examining veto designed for clinical use.

In vivo Function of Engineered Veto Cells

It was determined whether engineered veto functioned in the animal. C57BL/6 (H-2^{b})-derived fibroblasts transfected to express the CD8 α-chain were injected into Balb/c (H-2^{d}) mice. Control animals were injected with non-transfected fibroblasts. Spleen cells were harvested after 8 to 40 days and introduced into MLCs cultures with C57BL/6 (H-2^{b}) spleen cells as stimulator cells. After 5 days, cultures were harvested and tested for their ability to lyse EL4 (C57BL/6, H-2^{b}) target cells. Induction of anti-H-2^{b} CTL responses was completely suppressed in animals that had been injected with CD8 α-chain expressing fibroblasts (FIG. 4). Inhibition of anti-H-2^{b} T cells was highly specific. T cells from these mice still mounted responses to third party H-2^{k} allo-MHC molecules. These experiments confirmed that engineered veto cells specifically suppressed immune responses in vivo similar to conventional veto cells and that non-classical veto cells could be engineered to become veto cells. In other words, engineered cells negatively immunized animals to antigens carried on these cells.

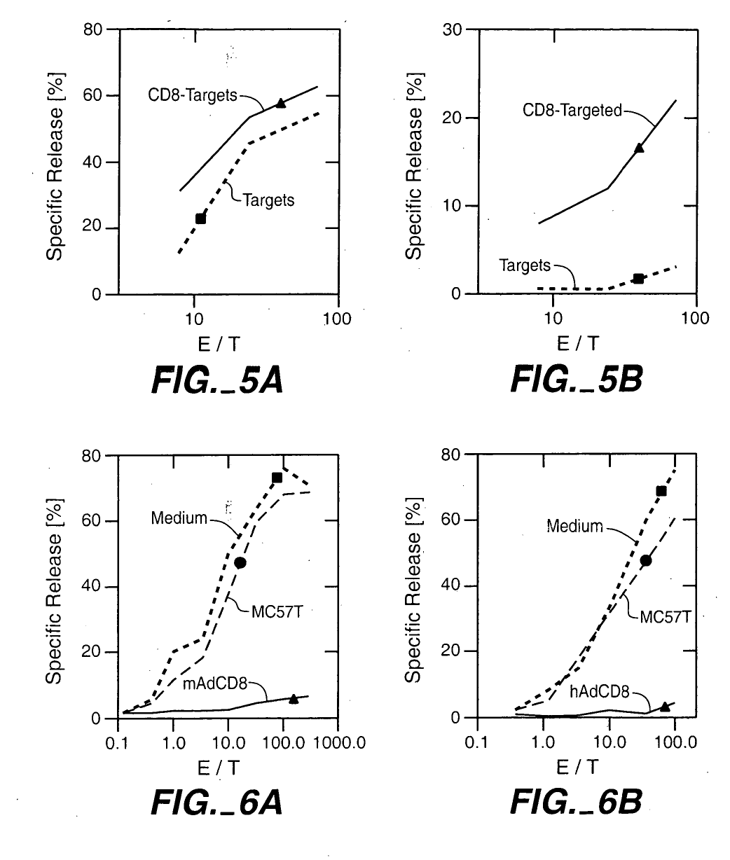

It was tested whether expression of the CD8 α-chain interfered with the function of fully activated T cells. For this purpose, target cells expressing CD8 α-chains were tested for their susceptibility to lysis by fully activated CTLs. Two different T cell populations were chosen for these studies, allo-reactive CTLs stimulated in a MLCs and activated peptide-specific CTLs. As depicted in FIG. 5, targets expressing the CD8 α-chain were lysed efficiently by populations of alloreactive T cells, but not by antigen-specific T cells. These results suggested that engineered veto was able to interfere even with on-going antigen specific immune responses, such as those found in autoimmune responses.

b. Viral Transfer Vectors to Engineer Fibroblasts as Veto Cells

Veto function of the Adenoviral Transfer Vector m-CD8: A replication-deficient vector Adenoviral Transfer Vector (mAdCD8α) was developed that carried the mouse CD8 α-chain. Mouse fibroblasts (MC57) that had been infected with the mAdCDB veto transfer vector expressed high levels of the mouse CD8 α-chain on day 2. In these fast proliferating cells, expression of the mouse CD8 α-chain is significantly reduced by day 5. mAdCD8 also infected other mouse cell lines, such as EL4, albeit with lower efficiency (data not shown).

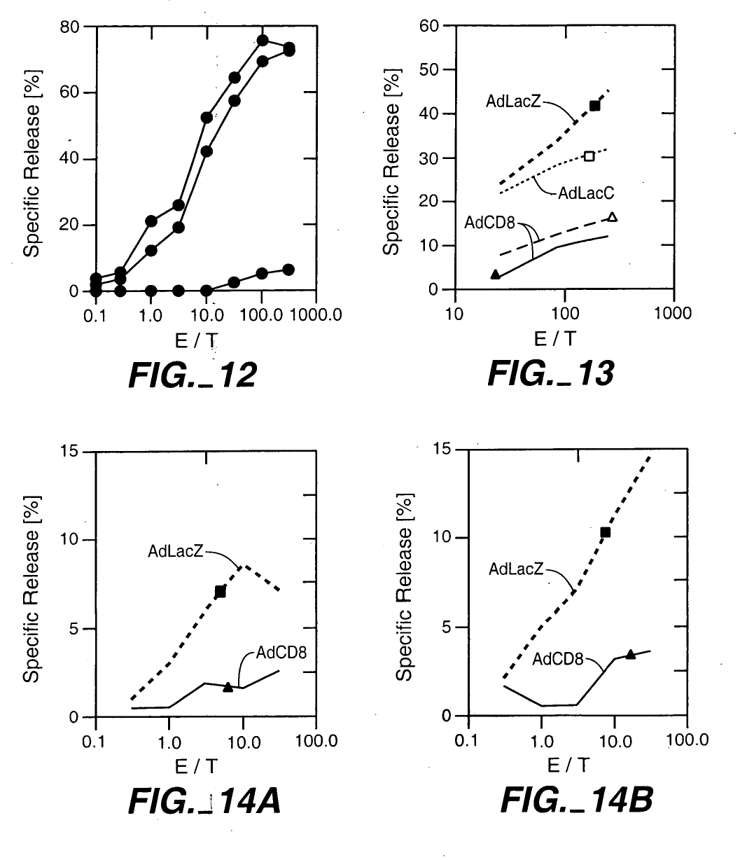

In subsequent experiments, mAdCD8 α-infected MC57 fibroblasts (H-2^{b}) were added to Balb/C (H-2^{d}) anti-C57B1/6 (H-2^{b}) MLCs. After 5 days, the cultures were harvested and tested for the presence of anti-H-2^{b} CTLs. MLCs to which infected fibroblasts had been added, no longer contained anti-H-2^{b} CTLs (FIG. 12). These experiments established the ability of a veto transfer vector to mediate immune suppression.

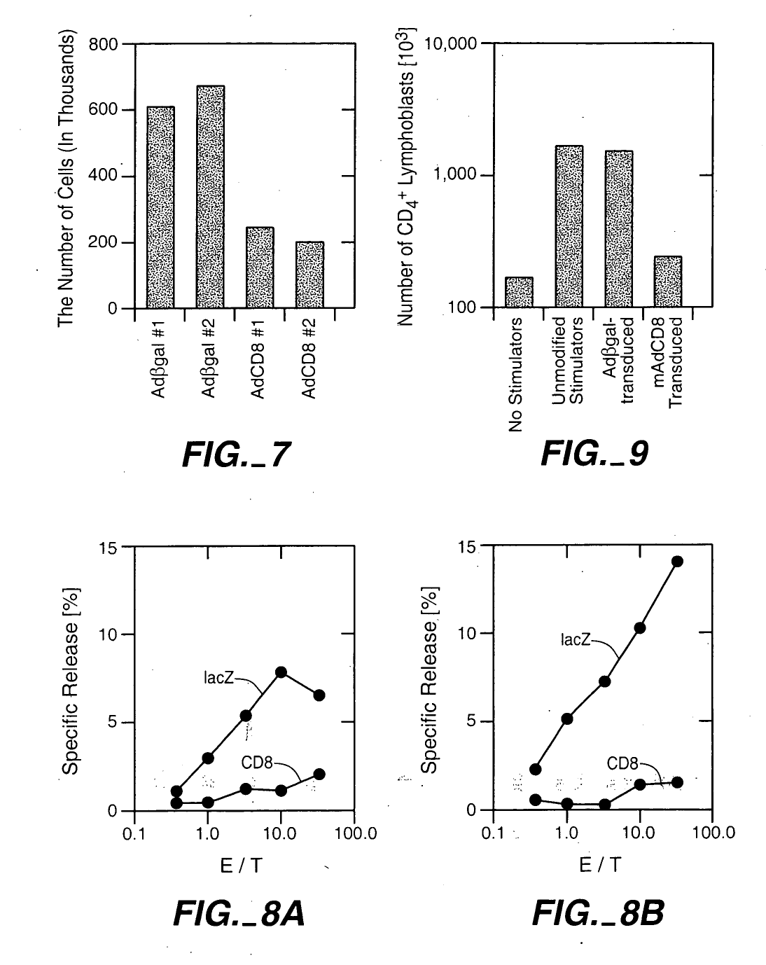

In addition, the human CD8-version of the Adenoviral vectors have been produced. Also, Adenoviral Associated Viruses that expressed mouse CD8 α-chain have been produced. It has been demonstrated that these viruses induce expression of the respective CD8 chains. Adenoviral veto vectors expressing either the mouse or the human CD8 α-chain mediated the complete inhibition of the induction of killer T cells (see FIG. 7).

Negative immunization with the mAdCD8 Veto Transfer Vector: Two different experiments were set up to determine whether mAdCD8 suppressed immune responses in vivo. In the first experiment, C57Bl/6 mice were infected with equivalent doses of either the mAdCD8 veto transfer vector or a similar adenoviral control vector coding for β-galactosidase, instead of the mouse CD8 α-chain (Adβgal). Seven days after immunization, these animals were sacrificed. Single cell suspensions of their spleen cells were cultured in the presence of Adβgal viruses for 5 days. Then the cultures were harvested and their ability to proliferate was evaluated. As depicted in FIG. 7, T cells proliferated vigorously to Adβgal harvested from mice immunized with Adβgal indicative of the presence of the highly proliferative CD4+ T cells. In contrast, T cells harvested from mAdCD8-injected animals failed to expand.

In a second step, we tested whether these cultures contained functional CD8⁺ CTLs testing them for their ability to lyse Adβgal-infected target cells (EL4, H-2^{b}). CTLs could only be revealed in cultures established form mice injected with Adβgal (FIG. 8). This first experiment suggested that AdCD8α did not induce responses to the adenoviral antigens possibly due to the expression of the CD8 α-chain. However, it was possible that AdCD8 failed to induce immune responses for different reasons. AdCD8 was non-functional in some undefined way, or the mice could only react with the β-galactosidase protein not found in mAdCD8.

To test the validity of the different conclusions, C57Bl/6 mice were injected once with either mAdCD8 or Adβgal followed by a second infusion with Adβgal after 7 days. Seven days later, mice were sacrificed, and 5-day spleen cell cultures were established in the presence of Adβgal. The responding T cells were tested for their lytic ability towards Adβgal-infected target cells (FIG. 8). Indeed, two exposures to Adβgal led to improved immunization. These studies also showed that after an AdCD8 injection, mice no longer responded to Adβgal and that Adβgal primarily, if not exclusively induced CTL responses towards the adenoviral proteins common to both vectors. This set of experiments strongly suggests that it will be possible to produce a gene therapy viral vector able to negatively immunize against responses towards genes carried on these vectors.

Inhibition of CD4⁺ T lymphocytes by veto: To examine whether veto transfer vectors can be used to inhibit the induction of CD4+ T lymphocytes, the following experimental system was established. C57Bl/6-derived fibroblast stimulator were transformed to express an allogeneic MHC class II molecule (H-2E^{k}) and the immune stimulatory CD80. These slow-proliferating fibroblasts non-irradiated to preserve their full stimulatory capacity, were transduced with either the mAdCD8 or the Adβgal transfer vectors and added to unselected C57B1/6 spleen cells. After 4 days, these cultures were harvested and analyzed by surface immunofluorescence for the presence of activated, i.e. blasting, CD4⁺ T lymphocytes (FIG. 9). It was found that unselected C57B1/6 spleen cells cultured with normal or Adβgal-transduced stimulator cells had high numbers of CD4⁺ T lymphoblasts. In contrast, cultures to which mAdCD8-infected stimulators had been added, only few CD4+ T lymphoblasts were detected. These studies confirmed that veto inhibited CD4⁺ T lymphocytes and in addition that a viral veto transfer vector could be used for this purpose.

Surface Expression of the Mouse and Human CD8 α-Chains after Infection with the Different Virus Constructs

Staining Protocols:

mAdCD8:

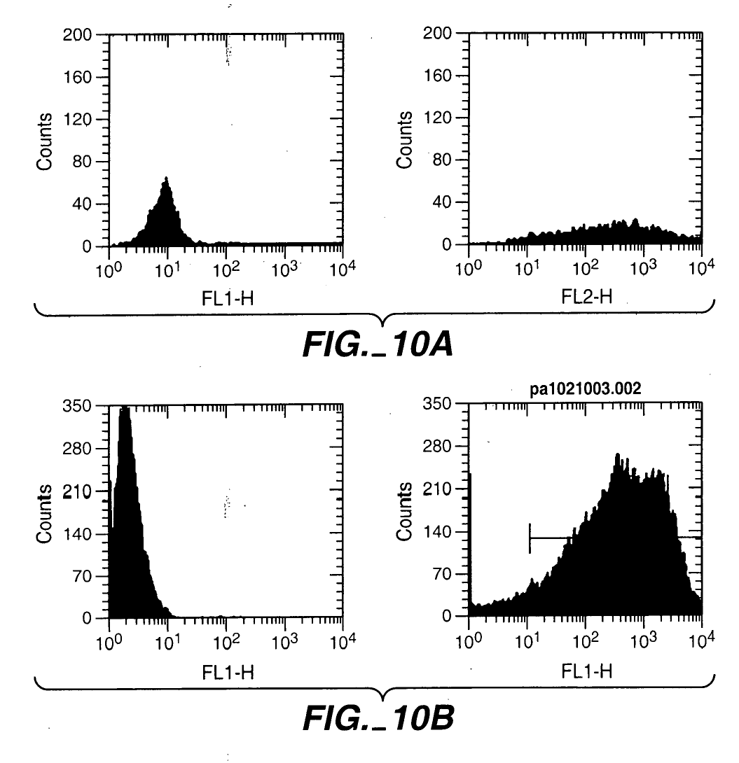

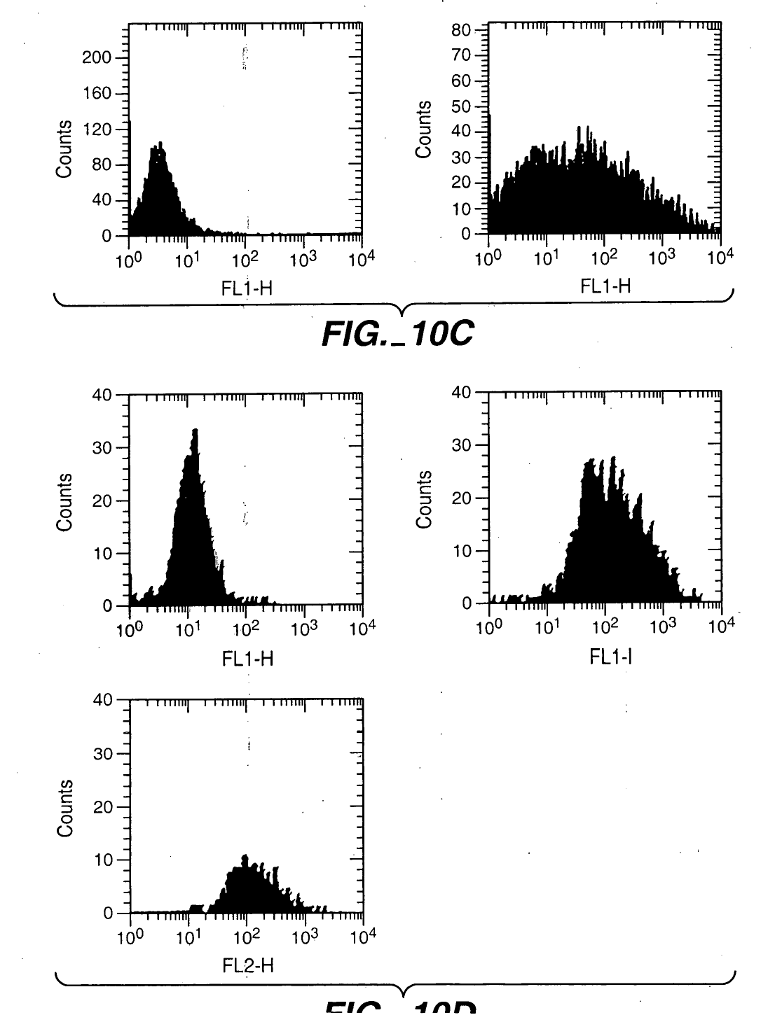

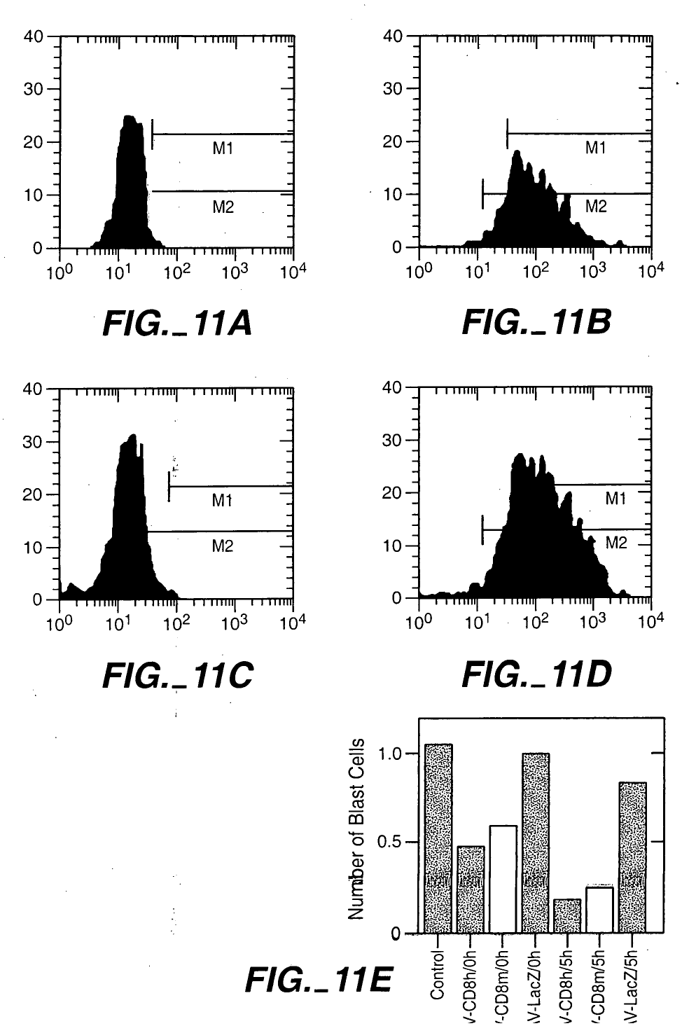

MC57T were mock-infected or infected with mAdCD8 at a multiplicity of infection of approximately 10⁴ for 3 days in modified IMDM. The infected cells were harvested and stained for the surface expression of the CD8 α-chain with the anti-mouse CD8 α-chain antibody directly labeled with FITC (Pharmingen). The extent of surface fluorescence was measured on a fluorescent activated cell analyzer (FACScan, Beckton-Dickinson) (FIG. 10).

Bone marrow cells were harvested from the cavity of femoral bones of Balb/c mice. The cells were infected with a β-galactosidase expressing. Adenoviral control vector (AdLacZ) or with mAdCD8 at a multiplicity of infection of 10⁴ for 3 days cultures in modified IMDM. The infected cells were harvested and stained for the surface expression of the CD8 α-chain with the anti-mouse CD8 α-chain antibody directly labeled with FITC. The extent of surface fluorescence was measured (FIG. 10C). In addition, it was determined that several cell types including CD34+ bone marrow cells, i.e. cells within the stem cell pool, were transduced efficiently (Table 1)

TABLE 1

| | | |
|---|---|---|
| Marker | Cell Type | Positive Staining |

| | | | |
|---|---|---|---|
| CD11a | Leukocytes | 29.3% | 31.5% |
| CD34 | Hematopoietic | 13.8% | 10.5% |
| | Lineages | | |
| CD19 | B Lymphocytes | 0.6% | 7.7% |
| CD3 | T Lymphocytes | 0.6% | nd |

hAdCD8:

MC57T were mock-infected. The viral titer of the hAdCD8 is not known. 100 μl of its stock solution was used to infect 3×10⁵ cells for 3 days. The infected cells were harvested and stained for the surface expression of the CD8 α-chain with the anti-human CD8 α-chain antibody directly labeled with FITC (Pharmingen). The extent of surface fluorescence was measured on a fluorescent activated cell analyzer (FIG. 10).

AAV-Based Veto Vectors

AAV-based veto vectors were produced in parallel using a Strategene/Avigen system. In these constructs, the human and mouse CD8 α-chains were driven from the same CMV intermediate early promotor/enhancer. The two viruses, mAAVCD8 and hAAVCD8 were packaged in the HEK 293 packaging cell line. The system employed is free of helper virus. mAAVCD8 and hAAVCD8 efficiently infected mouse fibroblasts (MC57T) and drove high levels of expression of the mouse or human CD8 α-chains, respectively. The extent of fluorescence was measured on a fluorescent activated cell analyzer (FIG. 10D). It is interesting to note that high levels of CD8 α-chain expression was seen within 36 hours after transduction. This finding was in contrast to observation by others. They had found that AAV-driven gene expression took several days to reach significant levels (PH Schmelck, PrimeBiotech). Additional studies with AAV veto vectors reiterated our previous findings that they could be used to suppress immune responses. Here, the standard MLC protocol was used (FIG. 6).

### EXAMPLE 2

### In vitro Inhibition Studies—Mixed Lymphocyte Cultures

Spleen cells were harvested from Balb/c (H-2^{d}) and C57BL/6 (H-2^{b}) mice. Single cell suspensions were prepared. The C57BL/6 spleen cells were irradiated with 3,000 rad (Mark 1 Cesium Irradiator). 4×10⁶ Balb/c spleen cells (responder/effector cells) were cultured together with 4×10⁶ irradiated C57BL/6 spleen cells (stimulator cells) per well in 24-well plates (TPP, Midwest Scientific, Inc.) in IMDM (Sigma) that contained 10% fetal calf serum (FCS) (Sigma), HEPES, penicillin G, streptomycin sulfate, gentamycine sulfate, L-glutamine, 2-mercaptoethanol, non-essential amino acids (Sigma), sodium pyruvate and sodium bicarbonate (modified IMDM). After 5 days of culture in a CO₂ incubator (Forma Scientific), the cultures were harvested in their entirety and tested for the ability to lyse C57BL/6-derived target cells (H-2^{b}).

To some of these cultures 4×10⁵ MC57T fibroblasts (H-2^{d}) were added that had been irradiated with 12,000 rad. In inhibition cultures, 4×10⁵ MC57T cells were included that had been infected with mAdCD8 at a multiplicity of infection of approximately 10⁴ to 1 for 2 days.

Cytotoxic T Lymphocyte Killer Assays

Cells harvested from the mixed lymphocyte cultures were counted for the number of blast cells, as an indicator of activated T lymphocytes. These effector cells were added to a single well in a U-bottomed 96-well plate. The number of effectors per well was titrated in 3-fold titration steps starting from 3×10⁶ or 1×10⁵ effectors per well. To these effector cells 1×10⁴ target cells EL4 (H-2^{b}), MC57T (H-2^{b}) or P815 (H-2^{d}) per well were added. The target cells had previously been labeled with ⁵¹Cr (Na-Chromate, Perkin-Elmer). 1×10⁶ target cells had been incubated with 100 μCi in a modified IMDM in a volume of approximately 500 μl for 90 min. Thereafter, the non-incorporated ⁵¹Cr was removed my multiple washes with modified IMDM.

The effector and target cells were incubated in a total volume of 200 μl for 4 hrs in a CO₂ incubator. Thereafter, the plates were spun in centrifuge (Centra CJ35R, International Equipment Company) at 1,500 rpm for 3 min. 100 ml of medium was removed from each well and the amount of ⁵¹Cr released from the target cells was counted in a Model 4000 Gamma counter (Beckman Instruments). Control cultures were set, in which effector cells were omitted to determine the background release. Total ⁵¹Cr incorporation into target cells was determined in wells, in which a 1% solution (w/v) of Triton X100 (Sigma) was substituted for the effector cells.

The amount of specific lysis was determined as:
in %=(specific release−background release)/(total release−background release)×100

The Activity of mAdCD8 in vitro

Mixed lymphocyte cultures were set up (Balb/c anti-C57BL/6). To these cultures MC57T fibroblasts were added (as indicated) that had been irradiated with 12,000 rad and had been infected with mAdCD8. After 5 days of culture, the cultures were harvested and tested for their ability to lyse EL4 (H-2^{b}) target cells at different effector-to-target (E/T) ratios (see FIG. 4).

As can be seen, even in the mixed lymphocyte culture, the cells expressing CD8 inhibited the induction of lytic T lymphocytes.

Production of mAdCD8 and hAdCD8

Both Adenoviral vectors were produced with the help of the AdEasy™ system from Biogene. Here the mouse and human CD8 α-chain cDNA is incorporated into the Transfer Vector (Step 1). Recombination with the Ad5ΔE1/ΔE3 vector is achieved in BJ5183 EC bacteria (Step 2). The recombinant vector is then transferred into the QBI-HEK 293A cells that contain the E1A and E1B Adenovirus 5 viral genes, which complement the deletion of this essential region in the recombinant adenovirus. The hAdCD8 and mAdCD8 produced in these cells are thus replication deficient.

As control vector expressing the bacterial LacZ gene (β-galactosidase) the Qbiogene provided QBI-Infect+ Viral Particle (Ad5.CMVLacZΔE1/ΔE3). Mouse CD8 a-chain sequence used. This sequence is similar to the published mouse sequence:

ACTUAL SEQUENCE: MASPLTRFLS LNLLLMGESI
ILGSGEAKPQAPELRIFPKK MDAELGQKVD LVCEVLGSVS QGCSWLFQNS
SSKLPQPTFVVYMASSHNKI TWDEKLNSSK LFSAVRDTNN KYVLTLNKFS
KENEGYYFCSVISNSVMYFS SVVPVLQKVN STTTKPVLRT PSPVHPTGTS
QPQRPEDCRPRGSVKGTGLD FACDIYIWAP LAGICVAPLL SLIITLICYH
RSRKRVCKCPRPLVRQEGKP RPSEKIV

Human CD8 α-chain sequence used. This sequence has a silent mutation compared to the published human sequence as indicated.

ACTUAL SEQUENCE: MALPVTALLL PLALLLHAAR
PSQFRVSPLDRTWNLGWTVE LKCQVLLSNP TSGCSWLFQP RGAAASPTFL
LYLSQNKPKAAEGLDTQRFS GKRLGDTFVL TLSDFRRENE GYYFCSALSN
SIMYFSHFVPVFLPAKPTTT PAPRPPTPAP TIASQPLSLR PEACRPAAGG
AGNRRRVCKCPRPVVKSGDK PSLARYV

Production of pAAV-mCD8 and pAAV-hCD8

These vectors were produced with the help of the AAV Helper-Free System from Stratagene. The system works by inserting the mouse and human sequences into the pAAV-MCS cloning vector. This plasmid is then co-transfected into HEK 293 cells together with a helper plasmid (containing the necessary Adenoviral proteins) and the pAAV-RC vector (containing the capsid genes) to produce the recombinant AAV particles.

### EXAMPLE 3

### Engineered Veto in Animal Models

Prior to the transplantation studies, we investigated how animals responded to the injection of large doses of the mAdCD8. In the first set of experiments, Balb/c mice (two mice in each group) were injected i.v. with equivalent doses of mAdCD8 or an Adenoviral control vector coding for β-galactosidase (AdLacZ). After seven days the animals were sacrificed. Their spleen cells were cultured in the presence of AdLacZ for five days. They were then tested for their ability to lyse AdLacZ-infected target cells (P815, Balb/c-derived). As depicted in FIG. 13, CTLs with specific lytic ability could be expanded from Balb/c mice that had been immunized with AdLacZ, but not from mice that had received the mAdCD8. This result suggested that AdCD8 did not induce immune responses to Adenoviral antigens due to the expression of the CD8 α-chain.

In a second set-up, C57Bl/6 mice were immunized with equivalent doses of mAdCD8 (2 mice) or AdLacZ (2 mice). Seven days after immunization, one animal of each group was sacrificed. Their spleen cells were cultured in cell suspension in the presence of AdLacZ for five days. They were then tested for their ability to specifically lyse AdLacZ-infected target cells (EL-4, C57Bl/6-derived). Again, injection of AdLacZ had induced the development of specific killer cells albeit at a low frequency, whereas mAdCD8 had failed to do so (FIG. 14).

In the second phase of this experiments, the remaining C57BL/6 mice that had received either mAdCD8 or AdLacZ received a second dose of AdLacZ seven days after their first viral injection. Seven days later, mice were sacrificed, and five-day spleen cell cultures were established in the presence of AdLacZ. The responding T cells were again tested for their lytic ability towards AdLacZ-infected EL4-target cells (FIG. 14B). Indeed, two exposures to AdLacZ led to a somewhat improved immunization. However, the animal that had previously received mAdCD8 still failed to mount a response. These experiments suggest that AdCD8 not only failed to induce immune responses, but prevented the induction immune responses directed against itself. Thus, mAdCD8 evaded the immune system.

Skin Transplantation

A skin transplantation model was set up. In the first step, mice were sacrificed and sections of their skins were harvested. They were infected with an Adenoviral control virus (AdLacZ) that carried β-galactosidase. Twenty-four hours after transduction these skin pieces were cultured in medium containing IPTG, which through the enzymatic action of the expressed β-galactosidase was converted into a blue dye (data not shown). In parallel to these studies, we optimized the surgical procedure. Donor animals were sacrificed and an approximately 0.5 cm² oval shaped piece of full-thickness back skin was harvested. Adipose tissue was carefully removed. In syngeneic transplantation studies Balb/c skin was transplanted onto Balb/c recipients. The skin healed well over time (data not shown). We then switched to a fully allogeneic strain combination, in which Balb/c donor skin infected either with mAdCD8 or AdLacZ was transplanted onto C57BL/6 mice. At day 12, all AdLacZ-infected skins showed evidence of active inflammation, whereas the mAdCD8-infected skins appeared healthy at this time point. In this model, however, tolerance was not induced and the skins eventually succumbed to rejection, apparently due to the later-determined fact that the majority of skin cells are refractory to infection with adenoviral vectors.

Allogeneic Heart Transplantation

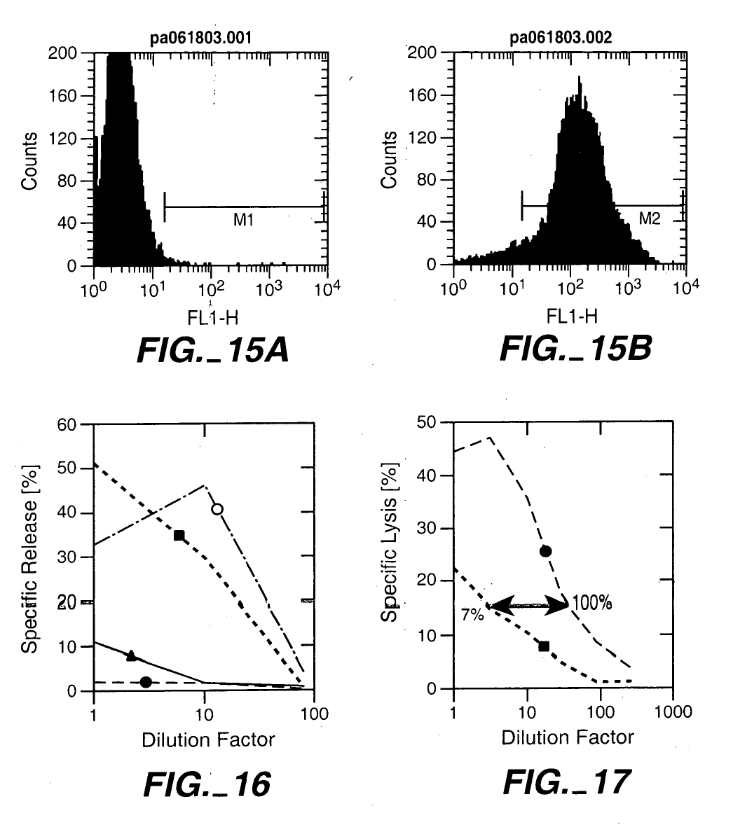

The following heart transplantation model was set up. Heart tissue of new-born mice was transplanted under the skin of the recipient mouse's ear. In parallel to establishing the transplantation system, we examined how heart muscle cells could be best infected with mAdCD8 vectors. Single cell suspensions from new-born hearts were prepared and infected with AdCD8 under different conditions. The extent of surface expression of the mouse CD8 α-chain was detected by immunofluorescence. As in FIG. 15, mAdCD8 very efficiently transduced mouse heart muscle cells after a 24 hour-incubation with mAdCD8 at an MOI of approximately 10³.

Having established the susceptibility of heart muscle cells to mAdCD8 transduction, we determined the concentration of virus, which was best suited to suppress the immune system. C57BlL/6 (H-2b) newborn hearts split in half, injected with 10⁹, 5×10⁷ or 10⁷ PFU of AdCD8 per graft and then incubated at 37° C. for four hours. Then they transplanted under the ear skin of fully allogeneic Balb/c (H-2d) mice. Mock-infected heart tissue was used in control experiments. Thirty-one days after transplantation, the mice were sacrificed and their spleens were harvested. MLCs (recipient-anti-donor (Balb/c anti-C57BL/6)) were set up for 5 days and tested for their ability to lyse C57BL/6-derived EL-4 target cells. As depicted in FIG. 17, T lymphocytes harvested from mice that received hearts either mock-infected or infected with the highest concentration of mAdCD8 showed high lytic responses towards cells of the donor-tissue type. T lymphocytes taken from mice that had received hearts infected with the two lower concentrations of mAdCD8 showed severely suppressed immune responses. In this test, an infection with 5×10⁷ PFU of mAdCD8 proved the most efficient. This virus amount was therefore used for next experiments. Additional Balb/c recipient mice were transplanted with C57Bl/6 hearts infected with the 5×10⁷ PFU of AdCD8 or mock-infected. One group of animals was sacrificed 38 days of the transplantation. The transplant-carrying ear was removed. The tissue was fixed and stained immunohistologically (anti-H-2b-Peroxidase/HE) for the presence of donor-type heart tissue. It was clear upon observation that only the AdCD8-infected heart tissue had survived. The heart tissue present did not show any evidence of cellular invasion indicating that rejection had been prevented. In the mice that had received mock-infected hearts, we could no longer discern intact heart tissue.

We also harvested the spleens from these animals and set up MLCs against irradiated spleen cells of donor-type. These cultures were tested for the presence of anti-C57BL/6 CTLs. As seen in FIG. 17, the lytic activity of T lymphocytes in the mAdCD8-mouse was reduced down to about 7% compared to T lymphocytes harvested from a control mouse. A second set of transplanted mice was kept for 52 days, at which time they were sacrificed and their grafts and immune activities were analyzed. In this study, the tissue was stained conventionally (HE) (data not shown) mAdCD8-infected heart tissue was healthy in appearance. In contrast, in animals that had received mock-infected hearts, muscle tissue again was barely discernable. The destruction of the tissue was accompanied by large numbers of invading cells.

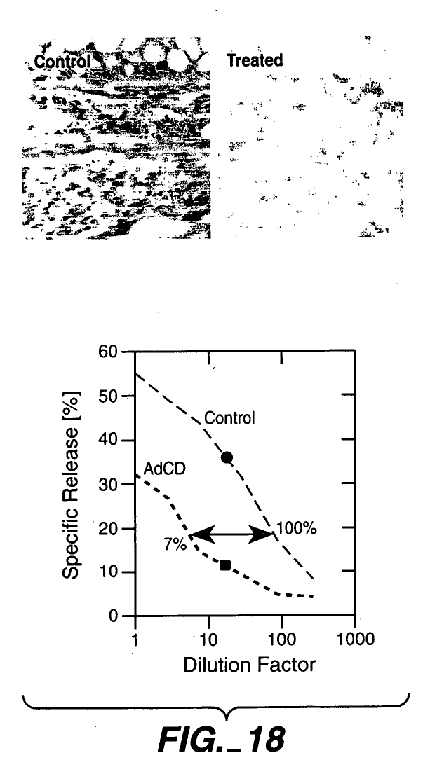

We again studied the function of alloreactive T cells in these animals. Spleen cells that have been harvested from the BALB/c recipients were stimulated with donor-type, i.e. C57BL/6, stimulator cells, then tested for their activity towards the stimulators. As illustrated in FIG. 18, the lytic activity of T lymphocytes from animals that had received AdCD8-treated hearts was significantly reduced to approximately 9% of control.

These experiments showed that mAdCD8 had not only prevented the rejection of the allogeneic heart, but had also significantly reduced the frequency of the respective alloreactive T lymphocytes. These experiments therefore indicated that engineered veto using mAdCD8 as the therapeutic vehicle could prevent the rejection of fully allogeneic organs.

Pancreatic Islet Transplantation

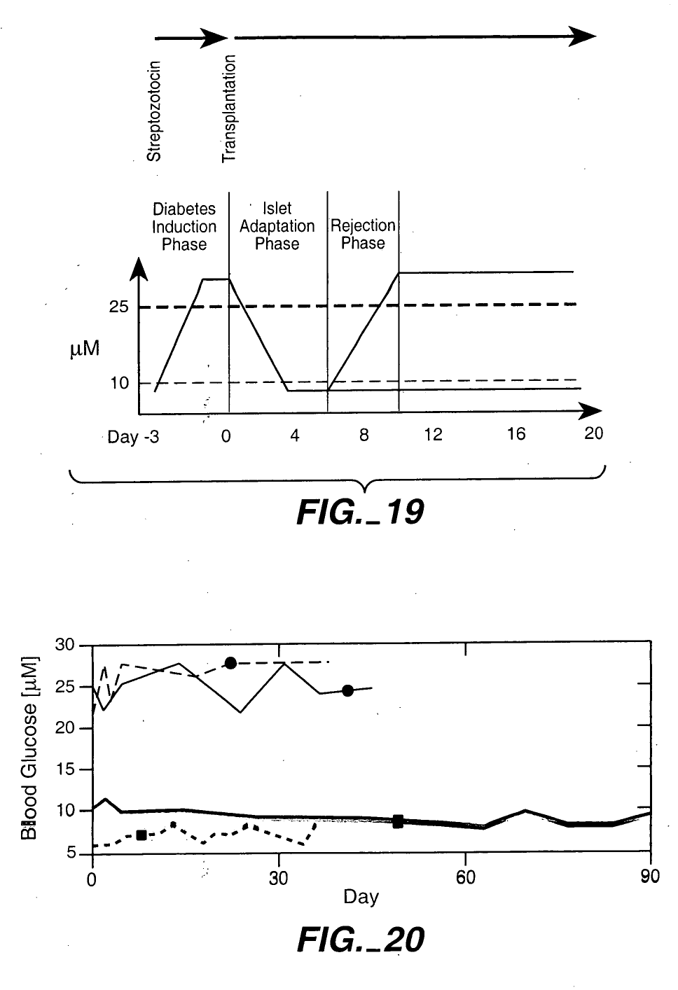

Both syngeneic and allogeneic transplantations were performed. Since transplant rejection commences approximately at approximately day 7, transplants were only scored if animals showed a reduction in the blood glucose levels during this initial period (see adaptation period in FIG. 19). It was assumed that the transplanted pancreatic islets were non-functional in animals, in which such an early blood glucose reduction (i.e. below 15 μM) was not seen. FIG. 20 depicts how blood glucose levels behave in untreated (i.e. normal) mice and in mice that had received streptozotocin to destroy their pancreatic islets.

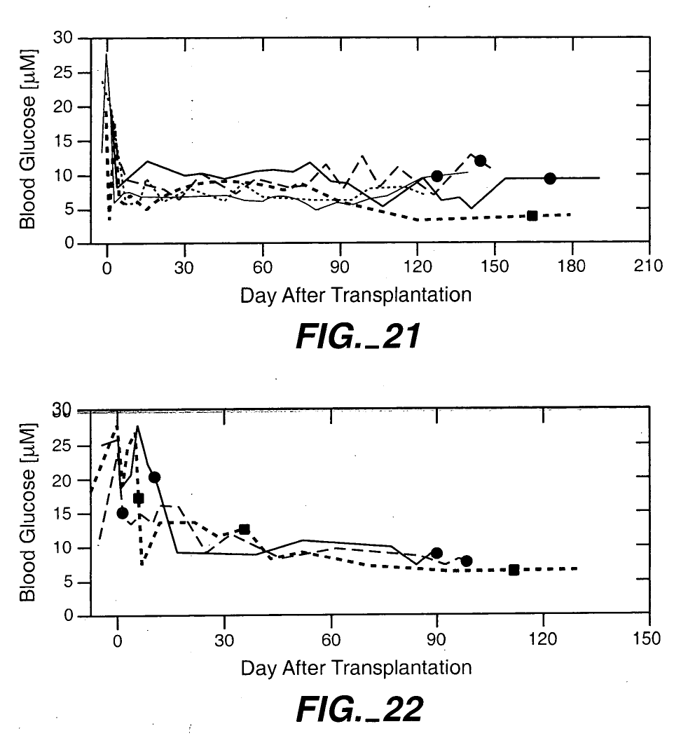

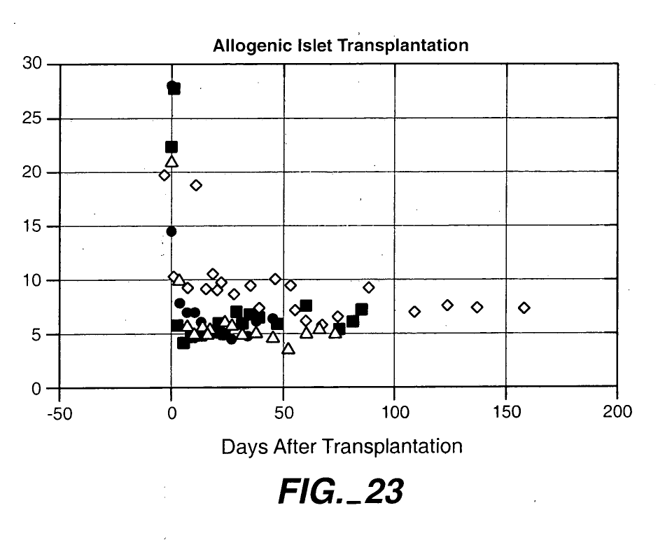

Two different strain combinations of syngeneic pancreatic islet transplantation were performed: C57BL/6 into C57BL/6 and Balb/c into Balb/c. In studies, the pancreatic islets had not been transduced. In both groups of mice, normal and stable glucose levels were achieved after transplantation. The entire observation period in these studies stretched to more than 6 months (FIG. 21). These studies were followed by experiments, in which the pancreatic islets had been transduced with mAdCD8. The infection conditions had been established previously (see above). Again, a normalization of blood glucose levels was seen (FIG. 21). Interestingly, in the case of Balb/c donors a delayed reduction of blood glucose levels was observed possibly indicating that transductions pancreatic islets took longer to adapt to their new environment.

It is well known that Adenoviruses and tissues transduced with Adenoviral vectors induce virulent immune responses leading to the destruction of the transduced tissue. This immunogenicity is of the major reasons why gene therapy has not been reaped the large benefits originally predicted. However, in our experiments infection of the pancreatic islets with an Adenoviral vector did not affect their survival in syngeneic hosts. Therefore, it had to be concluded that mAdCD8 had efficiently suppressed immune responses directed against Adenoviral proteins.

This idea had been independently supported when we had found that the veto vector mAdCD8 had evaded the immune system when directly injected into mice (see above). One of the Balb/c mice that had received a syngeneic mAdCD8-transduced transplant was sacrificed after 6 months. The transplanted pancreatic islets were identified under the kidney capsule and stained for the presence of the mouse CD8 α-chain. It was interesting to note that it could still be detected after 6 months (data not shown). This result would suggest that mAdCD8 had not been diluted out during this time probably due to an exceedingly low turnover rate of cells found in the pancreatic islets.

Next it was investigated whether mAdCD8 had been able to prevent the rejection of allogeneic pancreatic islets. The same experimental conditions were employed. Approximately 2,000 C57BL/6 pancreatic islets were transduced with mAdCD8 and transplanted into fully allogeneic Balb/c recipients. No additional immune suppressive therapy was used. It is obvious from these results that the initial phase of acute rejection was completely suppressed. Three of three mice did not show any signs of rejection within the first 30 days. However, even later stages of rejection, such as a chronic rejection, were efficiently inhibited. One of these mice had now been observed for more than 4 months without any evidence of a reduction in insulin production (FIG. 25). Therefore, we concluded from these still limited studies that engineered veto, here realized as an Adenoviral veto vector, prevented graft rejection in fully allogeneic strain combination.

Trachea and Lung Transplantation Model

We designed a series to studies to examine the ability of adenovirus carrying the mouse CD8 gene (AdCD8) to suppress the rejection of allogeneic transplants in a mouse model of tracheal and lung transplantation.

In our first study, tracheas were aseptically removed from C57Bl/6 donor mice, and freed of all attached tissue. These segments were infected with AdCD8 (1.2×10¹¹ pfu) for 24 hours, 37° C. These infected tracheal segments were then incubated with mouse anti-human CD8 FITC (Ancell Corp.). Non-infected tracheal segments were also incubated with these anti-sera. Tracheal fragments were examined microscopically under fluorescent light. The relative brightness of the samples indicates that human CD8 is present on the tracheal segments and is exposed, enabling the antibody to bind (Table 2). Incubation of tracheal segments with 1.8×10⁹ pfu of AdLacZ versus PBS showed that only the AdLacZ-treated sample became blue when incubated with the substrate IPTG.

TABLE 2

| | | Palpable | Relative |
|---|---|---|---|
| Treatment | Pfu | Transplant^{b} | Brightness^{a} |
| PBS | −− | −− | 50.72 |
| AdCD8 | 1.2 × 10⁹ | ++ | 90.58 |
| AdLacZ | 1.2 × 10⁹ | −− | (blue) |

Transplantation of AdCD8-treated mouse tracheal fragments. Five ring segments from BALB/c mice were freed of all connective tissue and infected overnight with 1.2×10¹¹ pfu of AdCD8 or 1.2×10⁹ pfu AdLacZ in IMDM . Samples were then placed under the ear skin of C57BAL/6 recipients. In a, samples were observed 24 hrs after infection. In b, samples were observed 60 days after transplantation.

PBS-treated control tissues were transplanted under the ear skin of Balb/c mice. No rejection of the vector-treated tissues has been observed to date and all transplanted segments are both visible and palpable. Transplants have been retained for 60 days.

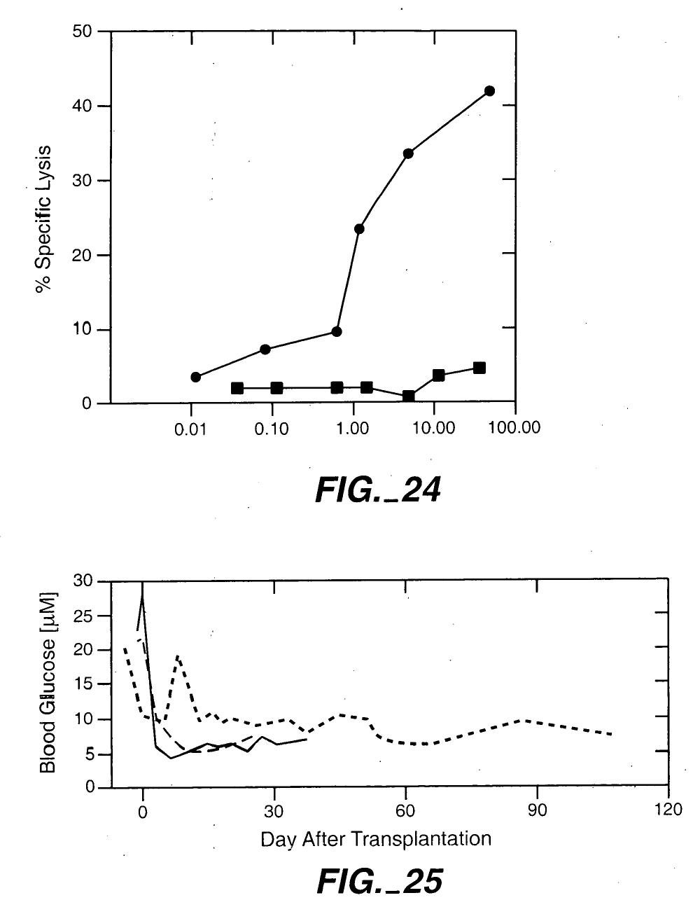

Samples of lung tissue from Balb/c donors were infected overnight with AdCD8 at 37° C. Tissues were then transplanted under the dorsal skin of C57BL/6 recipients. Control animals received non-treated or infected tissues. Animals were sacrificed at days 8, 12, 19, 20 and 22 following transplantation. Spleens were harvested to follow the immune response of the recipient in the presence of tissue with the vetoing CD8 molecule (FIG. 24). Similar to our previous findings in the heart transplantation model, treatment of the graft with AdCD8 had resulted in the suppression of transplant-specific CTLs in an assay designed to recognize allogeneic targets.

Samples of transplanted tissue were prepared for histology by fixation in formalin and staining with hematoxylin-eosin. The tissues from mouse lung that received no pre-treatment of AdCD8 (A) showed the classic pattern of cellular infiltration into the tissues as well as bronchi that were totally occluded by cells or collagen deposition (Genden, 2003).

Tissues that were pre-treated overnight with the vector had fewer bronchi that were infiltrated. At 22 days after transplant, lung tissue without vector treatment had cellular infiltration throughout the lung as well as in the bronchi and alveoli. Vector-treated tissue had far fewer areas of infiltration. The slides were evaluated by counting the number of open or closed airways in each group of samples. Tissues from non-treated lung tissue had 83% of the airways filled, and 17% of them open. In contrast, AdCD8-treated tissue had 30% of the tissues filled with cellular deposits and 76% of them open.

In conclusion, pre-treatment of tracheal and lung tissues with AdCD8 did significantly reduce the appearance of inflammatory cells in the bronchi or alveolar passages in the recipient mice. It was not expected that this would be the case. Significant reduction in the histiological signs of OB is a major breakthrough in developing AdCD8 for therapeutic applications for lung transplant patients.

1. A method for specifically inhibiting a host immune response to target cell-specific antigens, comprising contacting a target cell expressing said antigen with an expression vector encoding a CD8 polypeptide comprising the CD8 α-chain, wherein said CD8 polypeptide is expressed by said target cell and whereby a host immune response against said target cell is specifically inhibited.

2. The method according to claim 1, wherein said target cell-specific antigen is an alloantigen.

3. The method according to claim 2, wherein said alloantigen comprises a donor alloantigen and said target cell comprises a donor allograft cell.

4. The method according to claim 2, wherein said alloantigen comprises a recipient antigen and said target cell comprises a recipient cell.

5. A method for specifically inhibiting immune responses to donor antigens in a recipient, comprising conditioning donor allograft cells having said donor antigens to express a CD8 polypeptide comprising the CD8 α-chain prior to or contemporaneous with transplantation of said allograft cells into said recipient, wherein expression of said CD8 polypeptide by said allograft cells specifically inhibits the recipient alloimmune response to said donor antigens.

6. A method for extending the survival of an allograft in a recipient, comprising conditioning cells of said allograft to express a CD8 polypeptide comprising the CD8 α-chain prior to or contemporaneous with transplantation of said allograft into said recipient, wherein the CD8 polypeptide is expressed by said allograft cells and whereby the survival time of said allograft is extended.

7. The method according to claim 5 or 6, wherein said conditioning step comprises contacting said allograft cells with an expression vector encoding said CD8 polypeptide.

8. The method according to claim 5 or 6, wherein said conditioning step occurs ex vivo prior to or contemporaneous with transplantation of said allograft cells into said recipient.

9. The method according to claim 5 or 6, wherein said conditioning step occurs in vivo in the donor prior to or contemporaneous with harvesting of said allograft cells.

10. A method for specifically inhibiting immune responses to recipient antigens by transplanted donor T cells, comprising conditioning recipient cells at risk of graft versus host disease to express a CD8 polypeptide comprising the CD8 α-chain contemporaneous with or subsequent to transplantation of said donor T cells into said recipient, wherein expression of said CD8 polypeptide by said recipient cells specifically inhibits the donor alloimmune response to said recipient antigens.

11. A method for suppressing GVHD in a recipient, comprising conditioning cells of said recipient at risk for GVHD to express a CD8 polypeptide comprising the CD8 α-chain contemporaneous with or subsequent to transplantation of an allograft into said recipient, wherein the CD8 polypeptide is expressed by said recipient cells and whereby the GVHD immune response raised against the recipient cells by donor T cells in said allograft is suppressed.

12. The method according to claim 10 or 11, wherein said conditioning step comprises contacting said allograft cells with an expression vector encoding said CD8 polypeptide.

13. The method according to claim 10 or 11, wherein said conditioning step occurs in vivo in the recipient contemporaneous with or subsequent to transplantation of said donor T cells.

14. The method according to any one of claims 1 to 13, wherein said CD8 polypeptide is a human CD8 polypeptide.

15. The method according to any one of claims 1 to 14, wherein said CD8 polypeptide consists essentially of the extracellular domain of the CD8 α-chain and a transmembrane domain.

16. The method according to any one of claims 1 to 14, wherein said CD8 polypeptide consists essentially of the Ig-like domain of the CD8 α-chain and a transmembrane domain.

17. The method according to claim 15 or 16, wherein said transmembrane domain is the CD8 α-chain transmembrane domain.

18. An improved transplant allograft comprising allograft cells modified to express a CD8 polypeptide comprising the CD8 α-chain, wherein said allograft is capable of effectively and specifically inhibiting a recipient immune response to alloantigens.

19. The improved transplant allograft of claim 18, wherein modification of said allograft cells is achieved using viral-mediated delivery of a nucleic acid encoding said CD8 polypeptide.

20. The improved transplant allograft according to claims 18 or 19, wherein said CD8 polypeptide is a human CD8 polypeptide.

21. An improved organ preservation solution comprising a vector comprising a nucleic acid encoding a CD8 polypeptide, said CD8 polypeptide comprising a CD8 α-chain.

22. The improved organ preservation solution according to claim 21, wherein said CD8 polypeptide is a human CD8 polypeptide.

23. The improved organ preservation solution according to claim 21 or 22, wherein said CD8 polypeptide consists essentially of the extracellular domain of the CD8 α-chain and a transmembrane domain.

24. The improved organ preservation solution according to any one of claims 21 to 23, wherein said transmembrane domain is the CD8 α-chain transmembrane domain.

25. The improved organ preservation solution according to claim 21, wherein said nucleic acid encoding said CD8 polypeptide comprises the sequence set forth in (SEQ ID NOS:27-28).

26. The improved organ preservation solution according to claim 21, wherein said CD8 polypeptide consists essentially of the sequence as set forth in (SEQ ID NOS:27-28).
